(19) United States
(12) Patent Application Publication
(10) Pub. No.: US 2010/0120081 A1
(43) Pub. Date: May 13, 2010

(54) HIGH SENSITIVITY PARAMETERS FOR THE DETECTION OF VITAMIN B12 AND/OR FOLATE DEFICIENCIES AND METHODS OF USE

(75) Inventors: Ramon Simon-Lopez, St. Cergue (CH); Elena Sukhacheva, Le Vaud (CH)

Correspondence Address: HOWSON & HOWSON LLP / BECKMAN COULTER, INC., 501 OFFICE CENTER DRIVE, SUITE 210, FORT WASHINGTON, PA, 19034, US

(73) Assignee: Beckman Coulter, Inc., Brea, CA (US)

(21) Appl. No.: 12/613,908

(22) Filed: Nov. 6, 2009

Related U.S. Application Data
(60) Provisional application No. 61/112,499, filed on Nov. 7, 2008.

(51) Int. Cl. C12Q 1/02

(52) U.S. Cl. 435/29

(57) ABSTRACT

Described herein are high sensitivity parameters useful for the detection of vitamin B12 and/or folate deficiencies. Methods of determining susceptibility for vitamin B12 and/or folate deficiency in a subject are also provided. Methods of determining the progress and assessment of treatment of these deficiencies are provided.

This application claims the benefit of the priority of U.S. provisional patent application No. 61/112,499, filed Nov. 7, 2008. This provisional application is incorporated by reference herein.

### BACKGROUND OF THE INVENTION

Absorption of vitamin B12 from food requires normal function of the stomach, pancreas, and small intestine. Stomach acid and enzymes free vitamin B12 from food, allowing it, to bind to other proteins called R proteins (Shane B., In: Stipanuk M, ed. Biochemical and Physiological Aspects of Human Nutrition. Philadelphia: W.B. Saunders Co.; 2000:483-518). In the alkaline environment of the small intestine, R proteins are degraded by pancreatic enzymes, freeing vitamin B12 to bind to intrinsic factor (IF), a protein secreted by specialized cells in the stomach. Receptors on the surface of the small intestine take up the IF-B12 complex only in the presence of calcium, which is supplied by the pancreas. Vitamin B12 can also be absorbed by passive diffusion, but this process is very inefficient—only about 1% absorption of the vitamin B12 dose is absorbed passively.

Vitamin B12 deficiency is estimated to affect 10%-15% of individuals over the age of 60 (Baik et al., Annu. Rev. Nutr., 19:357-377, 1999). The most common causes of vitamin B12 deficiency are: 1) an autoimmune condition known as pernicious anemia and 2) food-bound vitamin B12 malabsorption. Although both causes become more common with increasing age, they are separate conditions (Baik et al., Annu. Rev. Nutr., 19:357-377, 1999). Vitamin B12 malabsorption can be caused by overgrowth of bacteria in the small intestine, inflammatory bowel disease, fish tapeworm infection, drugs such as antacids or metformin, lack of instrinsic factor and decreased stomach acidity.

Other causes of vitamin B12 deficiency include surgical resection of the stomach or portions of the small intestine where receptors for the IF-B12 complex are located. Conditions affecting the small intestine, such as malabsorption syndromes (celiac disease and tropical sprue), may also result in vitamin B12 deficiency. Because the pancreas provides critical enzymes, as well as calcium, required for vitamin B12 absorption, pancreatic insufficiency may contribute to B12 deficiency. Since vitamin B12 is found only in foods of animal origin, a strict vegetarian (vegan) diet has resulted in cases of vitamin B12 deficiency. Alcoholics may experience reduced intestinal absorption of vitamin B12 (Carmel R., In: Shils M E, Shike M, Ross A C, Caballero B, Cousins R J, eds. Modern Nutrition in Health and Disease. Philadelphia: Lippincott Williams & Wilkins; 2006:482-497). Individuals with acquired immunodeficiency syndrome (AIDS) appear to be at increased risk of deficiency, possibly related to a failure of the IF-B12 receptor to take up the IF-B12 complex (Shane B., In: Stipanuk M, ed. Biochemical and Physiological Aspects of Human Nutrition. Philadelphia: W.B. Saunders Co.; 2000:483-518). Long-term use of stomach acid-reducing drugs has also been implicated in vitamin B12 deficiency.

According to Herbert (Am. J. Clin. Nutr. 1994; 59 (suppl.): 1213S-22S) the transition from a normal vitamin B12 status to vitamin B12 deficiency can be subdivided into four stages. The first stage is usually characterized by a reduced vitamin B12 concentration in the serum. In the second stage it is already possible to observe depletion and the onset of a reduction in the store of vitamin B12 in the cells, and in the third stage there is already a biochemical vitamin B12 deficiency with severe functional disorders such as defective erythropoiesis. The fourth stage is a clinically manifest vitamin B12 deficiency in which anemia and nerve damage may be present. Vitamin B12 deficiency in humans leads to pernicious anemia which is a form of megaloblastic anemia. The hematological symptoms of a vitamin B12 deficiency are similar to those of a folate deficiency.

Common causes of folate deficiency include undernutrition and alcoholism, particularly when combined. Alcohol consumed in large amounts interferes with the absorption and metabolism of folate. Malabsorption disorders interfere with absorption of folate. Certain anticonvulsants (such as phenytoin, phenobarbital, and drugs used to treat ulcerative colitis (such as sulfasalazine) decrease the absorption of this vitamin. Methotrexate (used to treat cancer and rheumatoid arthritis), triamterene (used to treat high blood pressure), metformin (used to treat diabetes), and trimethoprim-sulfamethoxazole (an antibiotic) also interfere with the metabolism of folate.

Folate deficiency leads to megaloblastic anemia in humans. However, as a result of the close linkage between folic acid metabolism and vitamin B12 metabolism, anemia may not only be caused by a primary deficiency in folic acid but also by a secondary folic acid deficiency caused by a vitamin B12 deficiency.

The classical method of screening for a vitamin B12 and/or folate deficiency in a sample comprises analyzing the mean corpuscular volume (MCV), or the measure of the average red blood cell volume that is reported as part of a standard complete blood count. In patients with anemia, the MCV measurement allows classification as either a microcytic anemia (MCV below normal range) or macrocytic anemia (MCV above normal range). The normal MCV range is typically 80-100 femtolitres (fl) (Medline Plus, Red blood cell indices). In pernicious anemia (macrocytic), MCV can range up to 150 fl. High MCV value(s) has been the traditional criterion for detecting folate and vitamin B12 deficiencies. However, an MCV from a standard complete blood count within the normal range can mask existing or progressive B12 and/or folate deficiencies before a patient's clinical symptoms prompt a physician to order the more expensive, direct tests for B12 and/or folate levels.

Accurate and early diagnosis or detection of vitamin B12 and folic acid deficiencies in warm-blooded animals is important because these deficiencies can lead to life-threatening hematological abnormalities which may be reversible by treatment with vitamin B12 or folic acid, respectively. Thus, there exists a need in the art to develop more accurate and efficient methods for detection of these deficiencies.

### SUMMARY OF THE INVENTION

In one aspect, a method of detecting vitamin B12 and/or folate deficiency or monitoring the progress of treatment of these deficiencies, in a blood sample from a mammalian subject includes comparing a cell volume parameter of a test blood sample from the mammalian subject to a predetermined criterion, the cell volume parameter being obtained from a cell volume distribution of a white blood cell population in the test blood sample. In one embodiment a vitamin B12 and/or folate deficiency detected by this method can be coupled with anemia or can be present without clinical symptoms of anemia. In certain embodiments, the therapeutic efficacy or progress of treatment of these deficiencies, in the subject can also be assessed by these methods. Such deficiency or therapeutic efficacy is indicated by the difference in the cell volume parameter and the predetermined criterion.

In certain embodiments, the predetermined criterion is a cell volume parameter obtained from a white blood cell population of a plurality of control blood samples from the following groups of subjects, e.g., (a) subjects known to be deficient in vitamin B12 and/or folate; (b) healthy subjects; (c) subjects having a disease or disorder related to deficiencies in vitamin B12 and/or folate; and (d) a temporally-earlier test blood sample of the same test subject. It should be understood that when used throughout the specification, the term “predetermined criterion” and the control population referenced thereby are to be interpreted with the understanding that deficiencies in B12 and/or folate are indicated generally by cell volume parameters (e.g., MMOV and MNEV levels) that are statistically higher than levels of that parameter in normal, healthy subjects.

In one embodiment, a deficiency is diagnosed or detected when the cell volume parameter is statistically similar to or exceeds the predetermined criterion of (a) or (c). In another embodiment, a deficiency is diagnosed or detected when the cell volume parameter is statistically different from or exceeds the predetermined criterion of (b). In still another embodiment, a latent deficiency, i.e., without clinical signs of anemia, is indicated when the cell volume parameter is statistically different from the predetermined criterion of (b) and approaching the predetermined criterion of (a) or (c). In one embodiment, such a parameter may exceed (b) but be lower than (a) or (c). In yet another embodiment, the therapeutic efficacy is indicated when the difference in the test cell volume parameter and (b) is statistically closer than the difference in (d) and (b). For example, (d) is closer to (a) or (c) than it is to (b). Alternatively the test parameter is lower than (d). The opposite relationship applies for an unsuccessful treatment of the deficiencies.

In one aspect, the present invention is directed to methods of detecting a vitamin B12 and/or folate deficiency in a blood sample comprising the step of comparing a cell volume parameter of a test blood sample from a subject to a predetermined criterion, wherein the cell volume parameter is obtained from a cell volume distribution of a white blood cell population in the test blood sample. A vitamin B12 and/or folate deficiency in the individual is indicated when the cell volume parameter of the white blood cell population meets, approaches or exceeds the predetermined criterion from a population of B12 and/or folate deficient controls. Similarly a deficiency or progressive deficiency is indicated when the cell volume parameter of the subject exceeds the predetermined criterion derived from normal, healthy subjects.

In some embodiments, the subject is a mammal. In some embodiments, the subject is a human.

In another aspect, the invention provides a method of determining the likelihood that a subject has a vitamin B12 and/or folate deficiency comprising the step of comparing a cell volume parameter of a test blood sample from the subject to a predetermined criterion, wherein the cell volume parameter is obtained from a cell volume distribution of a white blood cell population in the test blood sample. A subject is likely to have a vitamin B12 and/or folate deficiency when the cell volume parameter of the white blood cell population exceeds the predetermined criterion from either a normal control population or a B12 and/or folate deficient population. Similarly a deficiency or progressive deficiency is indicated when the cell volume parameter of the subject meets or approaches the predetermined criterion derived the deficient population.

In another aspect, the invention provides a method of determining susceptibility for vitamin B12 and/or folate deficiency in a subject comprising the step of comparing a cell volume parameter for a test blood sample from the subject to a predetermined criterion, wherein the cell volume parameter is obtained from a cell volume distribution of a white blood cell population in the test blood sample. Susceptibility to a vitamin B12 and/or folate deficiency in the subject is indicated when the cell volume parameter approaches the predetermined criterion of the B12 and/or folate deficient population. The phrase “susceptibility for vitamin B12 and/or folate deficiency” as used herein refers to that a subject may not be identified as having a vitamin B12 and/or folate deficiency (i.e., the cell volume parameter in the test blood sample does not meet or exceed the predetermined criterion) but will develop a vitamin B12 and/or folate deficiency at a future date if not treated accordingly. For example, the cell volume parameter in the test blood sample of the subject may be near but not equal to or greater than the predetermined criterion of the B12 and/or folate deficient population, which indicates that the subject may be on the cusp of being deficient in vitamin B12 and/or folate without any clinical manifestation.

In another aspect, the invention provides a method of detecting vitamin B12 and/or folate deficiency with no clinical signs of anemia (i.e., latent B12 and/or folate deficiency) in a subject comprising the step of comparing a cell volume parameter for a test blood sample from the subject to a predetermined criterion, wherein the cell volume parameter is obtained from a cell volume distribution of a white blood cell population in the test blood sample. Latent vitamin B12 and/or folate deficiency in the subject is indicated when the cell volume parameter approaches the predetermined criterion. The term “latent vitamin B12 and/or folate deficiency” as used herein refers the condition of a subject that is not be identified as having a vitamin B12 and/or folate deficiency because the subject exhibits no clinical signs of anemia, but will develop anemia due to progressively worsening vitamin B12 and/or folate deficiency at a future date if not treated accordingly. The methods described herein are able to detect this latent deficiency with higher sensitivity and specificity than the existing methods, e.g., MCV.

In some embodiments, the methods described herein are performed by a computer processor or computer-programmed instrument that generates numerical or graphical data useful in detecting the deficiency.

In some embodiments, the white blood cell population is selected from the group consisting of myeloid cells, leukocytes, neutrophils and monocytes. In some embodiments, the white blood cell population comprises neutrophils. In other embodiments, the white blood cell population comprises monocytes. In still other embodiments, the white blood cell population comprises neutrophils and monocytes. The term “monocytes” as used herein includes monocytes, megaloblastic monocytes and other monocytic cells such as monoblasts, promonocytes, and myeloid related dendritic cells that may also be megaloblastic. The term of “neutrophils” as used herein includes neutrophils and megaloblastic neutrophils that are caused because the lack of Vitamin B12 and or folate deficiency. Megaloblastic neutrophils may have also a hypersegmented nucleus.

In various aspects, the cell volume parameter is selected from the group consisting of a mean of the cell volume distribution, a standard deviation of the cell volume distribution, an impedance measurement, a low angle light scatter measurement and an axial light loss measurement.

In certain embodiments, the method further comprises mixing the blood sample with a lytic reagent system prior to the comparing step.

In other embodiments, the predetermined criterion is a cell volume parameter obtained from a white blood cell population of a plurality of control blood samples from subjects known to be deficient in vitamin B12 and/or folate.

In other embodiments, the methods described herein further comprise (a) comparing a cell volume parameter obtained from a cell volume distribution of a first white blood cell subpopulation comprising neutrophils to a predetermined criterion obtained from a white blood subpopulation of neutrophils from a plurality of control blood samples from subjects known to be deficient in vitamin B12 and/or folate, and (b) comparing a cell volume parameter obtained from a cell volume distribution of a second white blood cell population comprising monocytes to a predetermined criterion obtained from a white blood cell subpopulation of monocytes from a plurality of control blood samples from subjects known to be deficient in vitamin B12 and/or folate.

In some embodiments, the methods described herein further comprise comparing a cell volume parameter obtained from a cell volume distribution of a red blood cell population in the test blood sample to a predetermined criterion obtained from a red blood cell population from a plurality of control blood samples from subjects known to be deficient in vitamin B12 and/or folate. A vitamin B12 and/or folate deficiency is detected when the cell volume parameter of the white blood cell population meets or exceeds the predetermined criterion obtained from a white blood cell population from a plurality of control blood samples from subjects known to be deficient in vitamin B12 and/or folate and when the cell volume parameter of the red blood cell population meets, approaches or exceeds the predetermined criterion obtained from a red blood cell population from a plurality of control blood samples from subjects known to be deficient in vitamin B12 and/or folate.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document.

In addition to the foregoing, the invention includes, as an additional aspect, all embodiments of the invention narrower in scope in any way than the variations defined by specific paragraphs herein. For example, certain aspects of the invention are described as a genus; and it should be understood that every member of a genus is, individually, an aspect of the invention. Also for all discussions of a genus, or a member of a genus, it should be understood that such terms embrace combinations of two or more members of the genus.

It should be understood that while various embodiments in the specification are presented using “comprising” language, under various circumstances, a related embodiment is also be described using “consisting of” or “consisting essentially of” language. It is to be noted that the term “a” or “an”, refers to one or more, for example, “an immunoglobulin molecule,” is understood to represent one or more immunoglobulin molecules. As such, the terms “a” (or “an”), “one or more,” and “at least one” is used interchangeably herein.

### BRIEF DESCRIPTION OF THE DRAWINGS

FIG. 1A shows an ROC curve comparing normal controls with B12 deficient subjects using mean neutrophil volume (MNEV or nedcmn).

FIG. 1B was based on FIG. 1A, but changes in the figure and in the calculations were based on more strict selection of patients for each group. All patients on treatment were excluded from normals and from the groups with other anemias. FIG. 1B shows receiver operator characteristic (ROC) curve comparing normal controls with B12 deficient subjects obtained as described in Example 1 using mean neutrophil volume (MNEV or the machine acronym, nedcmn). The ROC curve plots sensitivity versus 100-specificity, wherein the greater deviation from the x,y identity line indicated a greater diagnostic utility. The area under the curve is indicative of the use of the MNEV to distinguish subjects deficient in vitamin B12 from normal subjects or subjects with anemias not caused by the vitamin B12 deficiency.

FIG. 1C shows an ROC curve comparing normal controls with B12 deficient subjects using mean neutrophil volume (MMOV or the machine acronym, modcmn). The ROC curve plots sensitivity versus 100-specificity, wherein the greater deviation from the x,y identity line indicated a greater diagnostic utility. The area under the curve is indicative of the use of the MMOV to distinguish subjects deficient in vitamin B12 from normal subjects or subjects with anemias not caused by the vitamin B12 deficiency.

FIG. 2A shows an ROC curve comparing normal controls with folate deficient subjects using mean neutrophil volume (MNEV or nedcmn).

FIG. 2B was based on FIG. 2A, but changes in the figure and in the calculations were based on more strict selection of patients for each group. All patients on treatment were excluded from normals and from the groups with other anemias. FIG. 2B shows an ROC curve comparing normal controls with folate deficient subjects using mean neutrophil volume (MNEV or nedcmn). These results are obtained as described in Example 1. The ROC curve plots sensitivity versus 100-specificity, wherein the greater deviation from the x,y identity line indicated a greater diagnostic utility. The area under the curve is indicative of the use of the MNEV to distinguish subjects deficient in folate from normal subjects or subjects with anemias not caused by the folate deficiency.

FIG. 2C shows an ROC curve comparing normal controls with folate deficient subjects obtained as described in Example 1 using mean monocyte volume (MMOV or modcmn). The ROC curve plots sensitivity versus 100-specificity, wherein the greater deviation from the x,y identity line indicated a greater diagnostic utility. The area under the curve is indicative of the use of the MNEV to distinguish subjects deficient in folate from normal subjects or subjects with anemias not caused by the folate deficiency.

FIG. 3A shows an ROC curve comparing subjects with B12 or folate deficiency with other subjects with anemia using mean monocyte volume (MMOV or modcmn).

FIG. 3B shows an ROC curve comparing subjects with B12 or folate deficiency with other subjects with anemia obtained as described in Example 1 using mean neutrophil volume (MNEV or nedcmn). The ROC curve plots sensitivity versus 100-specificity, wherein the greater deviation from the x,y identity line indicated a greater diagnostic utility. The area under the curve is indicative of the use of the MNEV to distinguish subjects deficient in B12 and/or folate from subjects with anemias not caused by the folate or B12 deficiencies.

FIG. 3C was based on FIG. 3A, but changes in the figure and in the calculations were based on more strict selection of patients for each group. All patients on treatment were excluded from normals and from the groups with other anemias. FIG. 3C shows an ROC curve comparing subjects with 1312 or folate deficiency with other subjects with anemia obtained as described in Example 1 using mean monocyte volume (MMOV or modcmn). The ROC curve plots sensitivity versus 100-specificity, wherein the greater deviation from the x,y identity line indicated a greater diagnostic utility. The area under the curve is indicative of the use of the MMOV to distinguish subjects deficient in B12 and/or folate from subjects with anemias not caused by the folate or B12 deficiencies

FIG. 4A shows an ROC curve comparing subjects with B12 deficiency with other subjects with anemia using mean monocyte volume (MMOV or modcmn).

FIG. 4B shows an ROC curve comparing subjects with B12 deficiency with other subjects with anemia obtained as described in Example 1 using mean neutrophil volume (MNEV or nedcmn). The ROC curve plots sensitivity versus 100-specificity, wherein the greater deviation from the x,y identity line indicated a greater diagnostic utility. The area under the curve is indicative of the use of the MNEV to distinguish subjects deficient in B12 from subjects with anemias not caused by the B12 deficiency.

FIG. 4C was based on FIG. 4A, but changes in the figure and in the calculations were based on more strict selection of patients for each group. All patients on treatment were excluded from normals and from the groups with other anemias. FIG. 4C shows an ROC curve comparing subjects with B12 deficiency with other subjects with anemia obtained as described in Example 1 using mean monocyte volume (MMOV or modcmn). The ROC curve plots sensitivity versus 100-specificity, wherein the greater deviation from the x,y identity line indicated a greater diagnostic utility. The area under the curve is indicative of the use of the MMOV to distinguish subjects deficient in B12 from subjects with anemias not caused by the B12 deficiency.

FIG. 5A shows an ROC curve comparing subjects with folate deficiency with other subjects with anemia using mean monocyte volume (MMOV or modcmn).

FIG. 5B shows an ROC curve comparing subjects with folate deficiency with other subjects with anemia obtained as described in Example 1 using mean neutrophil volume (MNEV or nedcmn). The ROC curve plots sensitivity versus 100-specificity, wherein the greater deviation from the x,y identity line indicated a greater diagnostic utility. The area under the curve is indicative of the use of the MNEV to distinguish subjects deficient in folate from subjects with anemias not caused by the folate deficiency

FIG. 5C was based on FIG. 5A, but changes in the figure and in the calculations were based on more strict selection of patients for each group. All patients on treatment were excluded from normals and from the groups with other anemias. FIG. 5C shows an ROC curve comparing subjects with folate deficiency with other subjects with anemia obtained as described in Example 1 using mean monocyte volume (MMOV or modcmn). The ROC curve plots sensitivity versus 100-specificity, wherein the greater deviation from the x,y identity line indicated a greater diagnostic utility. The area under the curve is indicative of the use of the MMOV to distinguish subjects deficient in folate from subjects with anemias not caused by the folate deficiency.

FIG. 6A provides a Box and Whisker Plot graph comparing normal controls with B12 deficient subjects using mean neutrophil volume (MNEV or nedcmn).

FIG. 6B provides a Box and Whisker Plot graph comparing normal controls (right side of graph) with B12 deficient subjects (left side of graph) using mean neutrophil volume (MNEV or nedcmn). FIG. 6B was based on FIG. 6A, but changes in the figure and in the calculations were based on more strict selection of patients for each group. All patients on treatment were excluded from normals and from the groups with other anemias.

FIG. 6C provides a Box and Whisker Plot graph comparing normal controls (right side of graph) with B12 deficient subjects (left side of graph) using mean monocyte volume (MMOV or modcmn).

FIG. 7A provides a Box and Whisker Plot graph comparing normal controls with folate deficient subjects using mean neutrophil volume (MNEV or nedcmn).

FIG. 7B provides a Box and Whisker Plot graph comparing normal controls (right side of graph) with folate deficient subjects (left side of graph) using mean neutrophil volume (MNEV or nedcmn). FIG. 7B was based on FIG. 7A, but changes in the figure and in the calculations were based on more strict selection of patients for each group. All patients on treatment were excluded from normals and from the groups with other anemias.

FIG. 7C provides a Box and Whisker Plot graph comparing normal controls (right side of graph) with folate deficient subjects (left side of graph) using mean monocyte volume (MMOV or modcmn).

FIG. 8A provides a Box and Whisker Plot graph comparing subjects deficient in B12 or folate to other subjects with anemia using mean monocyte volume (MMOV or modcmn).

FIG. 8B provides a Box and Whisker Plot graph comparing subjects deficient in B12 or folate (left side of graph) to other subjects with anemia (right side of graph) using mean monocyte volume (MMOV or modcmn). FIG. 8B was based on FIG. 8A, but changes in the figure and in the calculations were based on more strict selection of patients for each group. All patients on treatment were excluded from normals and from the groups with other anemias.

FIG. 8C provides a Box and Whisker Plot graph comparing subjects deficient in B12 or folate (left side of graph) to other subjects with anemia (right side of graph) using mean neutrophil volume (MNEV or nedcmn).

FIG. 9A provides a Box and Whisker Plot graph comparing subjects deficient in B12 to other subjects with anemia using mean monocyte volume (MMOV or modcmn).

FIG. 9B provides a Box and Whisker Plot graph comparing subjects deficient in B12 (left side of graph) to other subjects with anemia (right side of graph) using mean monocyte volume (MMOV or modcmn). FIG. 9B was based on FIG. 9A, but changes in the figure and in the calculations were based on more strict selection of patients for each group. All patients on treatment were excluded from normals and from the groups with other anemias.

FIG. 9C provides a Box and Whisker Plot graph comparing subjects deficient in B12 (left side of graph) to other subjects with anemia (right side of graph) using mean neutrophil volume (MNEV or nedcmn).

FIG. 10A provides a Box and Whisker Plot graph comparing subjects deficient in folate to other subjects with anemia using mean monocyte volume (MMOV or modcmn).

FIG. 10B provides a Box and Whisker Plot graph comparing subjects deficient in folate (left side of graph) to other subjects with anemia (right side of graph) using mean monocyte volume (MMOV or modemn). FIG. 10B was based on FIG. 10A, but changes in the figure and in the calculations were based on more strict selection of patients for each group. All patients on treatment were excluded from normals and from the groups with other anemias.

FIG. 10C provides a Box and Whisker Plot graph comparing subjects deficient in folate (left side of graph) to other subjects with anemia (right side of graph) using mean neutrophil volume (MNEV or nedcmn).

FIGS. 11A, 11B and 11C provide VCS plots which demonstrate the mean volume of neutrophils in normal, B12 deficient and folate deficient samples. FIGS. 11D, E, and F were based on FIGS. 11A, 11B and 11C, but changes in the figure and in the calculations were based on more strict selection of patients for each group. All patients on treatment were excluded from normals and from the groups with other anemias.

FIG. 11D provides a volume conductivity scatter (VCS) plot which demonstrates the mean volume of neutrophils (MNEV) and mean volume of monocytes (MMOV) in a normal sample.

FIG. 11E provides a VCS plot which demonstrates the mean volume of neutrophils (MNEV) and mean volume of monocytes (MMOV) in a B12 deficient sample.

FIG. 11F provides a VCS plot which demonstrates the mean volume of neutrophils (MNEV) and mean volume of monocytes (MMOV) in a folate deficient sample.

FIG. 12A demonstrates that mean neutrophil volume in a sample decreases after treatment of a subject for a vitamin B12 deficiency.

FIG. 12 B is a graph showing the evolution of a patient with B12 deficiency with the classical MCV and the neutrophil volume (MNEV) from prior treatment. The graph demonstrates that mean neutrophil volume (MNEV) in a sample decreases after the treatment of a subject for a vitamin B12 deficiency. The correlation of the decrease of MCV and MNEV is 0.986. The regression equation is Y=25.2454+1.2514X, wherein Y is MNEV and X is MCV. The coefficient of determination R² is 0.9723. FIG. 12B is based on FIG. 12A, but changes in the figure and in the calculations were based on more strict selection of patients for each group. All patients on treatment were excluded from normals and from the groups with other anemias.

### DETAILED DESCRIPTION OF THE INVENTION

Described herein are high sensitivity parameters useful for the detection of vitamin B12 and/or folate deficiencies. As described herein, such deficiencies may be detected in subjects with anemia or without anemia (latent deficiency), and may be detected in the course of monitoring of the progress of treatment of such deficiencies. The inventors determined that an alteration in a cell volume parameter of a white blood cell population is more indicative of a vitamin B12 and/or folate deficiency than the classic method of determining such deficiencies (i.e., an alteration in a cell volume parameter, such as the mean corpuscular volume or MCV) of a red blood cell population). It is also shown that alterations in the cell volume parameters of both red and white blood cell populations more specifically identified the sample as being deficient in vitamin B12 and/or folate. Methods of the invention were developed after identifying specific cell parameters in samples from a population of patients known to suffer from a vitamin B12 deficiency and/or folate deficiency, and comparing these identified cell parameters to those in samples from a control population of patients known not to suffer from a vitamin B12 and/or folate deficiency. This control population of patients included two subpopulations, one that was known not to suffer from any form of anemia and one that suffered from anemia not associated with vitamin B12 and/or folate deficiency. Still other control populations are defined herein.

In one embodiment, a method of detecting a vitamin B12 and/or folate deficiency or monitoring the progress of the deficiency, in a blood sample from a mammalian subject comprises comparing a cell volume parameter of a test blood sample from the mammalian subject to a predetermined criterion. The cell volume parameter is obtained from a cell volume distribution of a white blood cell population in the test blood sample. A vitamin B12 deficiency and/or folate deficiency may be measured in a subject with or without clinical signs of anemia. Similarly, the therapeutic efficacy or progress of treatment of the test subject is indicated by the difference in the cell volume parameter and the predetermined criterion.

In one embodiment of such a method, the predetermined criterion is a cell volume parameter obtained from a white blood cell population of a plurality of control blood samples. In one embodiment such control samples are from subjects known to be deficient in vitamin B12 and/or folate. In another embodiment such control subjects are healthy subjects. In another embodiment, such control subjects are subjects having a disease or disorder related to deficiencies in vitamin B12 and/or folate. In another embodiment, the control sample is a temporally-earlier test blood sample of the same test subject. In still other embodiments, more than one predetermined criterion is employed in the method and thus, more than one of these categories of control subjects are used.

For example, in one embodiment of a method, a B12 and/or folate deficiency is diagnosed or detected when the cell volume parameter is statistically similar to the predetermined criterion derived from control samples from subjects known to be deficient in vitamin B12 and/or folate and/or derived from control samples from subjects having a disease or disorder related to deficiencies in vitamin B12 and/or folate.

In another embodiment, the method diagnoses or detects a B12 and/or folate deficiency when the cell volume parameter of the test subject is statistically different from the predetermined criterion derived from healthy subjects.

In still another embodiment, the method is used to detect a vitamin B12 and/or folate deficiency in a subject having no symptoms or clinical signs of anemia (Latent B12 and or Folate Deficiency). Latent deficiencies are detected when the cell volume parameter is statistically different from the predetermined criterion derived from healthy controls and is observed to be approaching the predetermined criterion derived from samples from subjects known to be deficient in vitamin B12 and/or folate or derived from samples from subjects having a disease or disorder related to deficiencies in vitamin B12 and/or folate.

In still another embodiment, the method is used to diagnose, detect or monitor the progress of a subject undergoing treatment for a B12 and/or folate deficiency. When the difference in the test cell volume parameter under evaluation and the predetermined criterion derived from healthy subjects is statistically closer than the difference in the white cell volume parameter of a temporally-earlier test blood sample of the same test subject and the predetermined criterion derived from healthy subjects, the treatment is adjudged successful. In certain embodiments, the converse of this relationship denotes an unsuccessful treatment of the deficiencies or diseases related thereto, e.g., anemia caused by the B12 and/or folate deficiency. Similarly if the subject's later cell volume parameter is closer to the predetermined criterion of the B12/folate deficient controls than it is to the predetermined criterion of the normal controls, and no difference is detected between the subject's later and earlier samples, then the therapy may be adjudged to be ineffective, and a progressive or latent deficiency is detected.

In other embodiments, the methods described herein include measuring the cell volume parameter obtained from a cell volume distribution of a white blood cell population in the test blood sample prior to the comparison. In still other embodiments, the methods described herein include measuring the cell volume parameter obtained from a cell volume distribution of a red blood cell population in the test blood sample prior to the comparison. In still other embodiments, the methods described herein include measuring other parameters obtained from the test blood sample prior to the comparisons described herein.

hi one embodiment, a method of this invention involves comparing a cell volume parameter obtained from a cell volume distribution of a first white blood cell subpopulation of neutrophils in the test sample to a first predetermined criterion obtained from a white blood cell subpopulation of neutrophils from a plurality of control blood samples; and comparing a cell volume parameter obtained from a cell volume distribution of second white blood cell subpopulation of monocytes in the test sample to a second predetermined criterion obtained from a white blood cell subpopulation of monocytes from a plurality of control blood samples.

In other embodiments, these methods can further include comparing a cell volume parameter obtained from a cell volume distribution of a red blood cell population in the test blood sample to a predetermined criterion obtained from a red blood cell population from a plurality of control blood samples. In such instances, the cell volume parameter of the red blood cell population is selected from the group consisting of a mean of the red blood cell volume distribution, a standard deviation of the red blood cell volume distribution, an impedance measurement, a low angle light scatter measurement and an axial light loss measurement, a mean of the cell volume distribution.

One aspect of the invention provides a method of detecting a vitamin B12 and/or folate deficiency in a blood sample comprising the step of comparing a cell volume parameter of a test blood sample from a subject to a predetermined criterion, wherein the cell volume parameter is obtained from a cell volume distribution of a white blood cell population in the test blood sample. A vitamin B12 and/or folate deficiency in the subject is indicated when the cell volume parameter meets, approaches or exceeds the predetermined criterion. In some embodiments, the white blood cell population is selected from the group consisting of myeloid cells, leukocytes, neutrophils and monocytes. In some embodiments, the white blood cell population comprises neutrophils. In some embodiments, the white blood cell population comprises monocytes. In some embodiments, the white blood cell population comprises both neutrophils and monocytes.

In another aspect, the invention provides a method of determining the likelihood that a subject has a vitamin B12 and/or folate deficiency comprising the step of comparing a cell volume parameter of a test blood sample from the subject to a predetermined criterion, wherein the cell volume parameter is obtained from a cell volume distribution of a white blood cell population in the test blood sample. A subject is likely to have a vitamin B12 and/or folate deficiency when the cell volume parameter of the white blood cell population meets, approaches or exceeds the predetermined criterion.

Another aspect of the invention provides a method of determining susceptibility for vitamin B12 and/or folate deficiency in a subject comprising the step of comparing a cell volume parameter for a test blood sample from the subject to a predetermined criterion, wherein the cell volume parameter is obtained from a cell volume distribution of a white blood cell population in the test blood sample. Susceptibility to a vitamin B12 and/or folate deficiency in the subject is indicated when the cell volume parameter meets, approaches or exceeds the predetermined criterion.

Another aspect of the invention provides a method of determining latent vitamin B12 and/or folate deficiency in a subject comprising the step of comparing a cell volume parameter for a test blood sample from the subject to a predetermined criterion, wherein the cell volume parameter is obtained from a cell volume distribution of a white blood cell population in the test blood sample. Latent vitamin B12 and/or folate deficiency in the subject is indicated when the cell volume parameter meets, approaches or exceeds the predetermined criterion.

The term “sample” as used herein includes a whole blood sample (i.e., includes any type of cell typically present in a blood sample such as red blood cells, and white blood cells). If desired, red blood cells and white blood cells within the whole blood sample are separated by any means known in the art including, for example and without limitation, cytochemical staining, impedance flow cytometry, volume conductivity scatter (VCS) methods, and the methods disclosed in co-owned U.S. Patent Publication No. 2008/0233553. Other leucocyte-containing samples may also be employed in this method, if desired.

The term “cell volume parameter” as used herein refers to a value associated with the cell volume distribution of a cell population (e.g., white blood cell or red blood cell population). In some embodiments, the cell volume parameter is measured by, for example, an impedance measurement (such as the mean or mean plus or minus one standard deviation of a cell population) as measured by either a direct current or radio frequency impedance measurement. The term “direct current impedance measurement” or “DC impedance measurement” as used herein refers to a value associated with the cell volume of a cell obtained by DC impedance. The term “radio frequency impedance measurement” as used herein refers to the intracellular contents of the cell. The term “cell distribution” or “cell distribution parameter” as used herein refers to a value obtained from an electronic or optical measurement as described herein.

In various aspects of the methods described herein, a cell volume parameter is defined as one or more of a mean of the cell volume distribution, a standard deviation of the cell volume distribution, an impedance measurement from the sample, a low angle light scatter measurement from the sample and an axial light loss measurement from the sample. For example, in one embodiment, the cell volume parameter is mean neutrophil volume, mean monocyte volume, or both parameters. In other embodiments, the mean cell volume of red blood cells is also used in these methods. It is anticipated that one of skill in the art can use any other conventional blood analysis method, or include other such methods with the specified steps defined by the methods herein.

When a particle or a blood cell, suspended in a conductive solution, passes through a flow cell or an aperture, an electrical signal, or a pulse, is measured due to the increase of impedance. The electrical pulses have been used for counting the number of blood cells of a blood sample. On the other hand, the pulse shape, height and width are directly related to the volume or size of a particle, and are converted to the volume of the cell being measured. When a sample that contains two or more different blood cells having different volumes is measured, a histogram obtained from the measurement represents volume distribution of these different blood cells. The detection methods and apparatus used for blood cell counting and sizing by a blood analyzer equipped with a DC impedance measurement device are generally described in U.S. Pat. Nos. 2,656,508; 3,810,011 and 5,125,737.

In some embodiments, the cell volume parameter of a cell population is determined using a volume conductivity scatter (VCS) measurement. VCS measurements are utilized on various commercial hematology analyzers, including those manufactured by Beckman Coulter, Inc. Fullerton, Calif. The term “VCS measurement” as used herein refers to a three-dimensional measurement technology which measures the direct current (DC) and radio frequency (RF) impedances, and light scatter signals of a blood cell when it passes through a flow cell. Among these three measurements, both DC impedance and RF impedance measurements are impedance measurements, which detect the increase of impedance as a cell carried in a conductive medium is passed through a flow cell. The VCS detection method has been described in detail in U.S. Pat. No. 5,125,737.

In some embodiments, the cell volume parameter is an average, or arithmetic mean, cell volume of a white blood cell population. For example, in some embodiments, the average cell volume parameter obtained from a white blood cell population is a mean cell volume of neutrophils in the sample. In such embodiments, for example and without limitation, a neutrophil mean cell volume (MNEV or nedcmn) of >140 channels indicates that the individual is deficient in vitamin B12 and/or folate. In other embodiments, the average cell volume parameter obtained from a white blood cell population is a mean cell volume of monocytes in the sample. In such embodiments, for example, without limitation, a monocyte mean cell volume (MMOV or modcmn) of >169 channels indicates that the individual is deficient in vitamin B12 and/or folate. Channels are directly proportional to the volume in femtoliters. Other levels of MNEV or MMOV may be used by one of skill in the art given the teachings herein, and depending upon the population forming the predetermined criterion as well as the individual subject(s). For example, some physiological characteristics of the subject, e.g., age, general health, weight, gender, race, and the like may result in higher or lower normal healthy averages, or abnormal, B12/folate deficient averages of MMOV or MNEV. One of skill in the art may adjust these measurement cutoffs while practicing the methods described herein.

In other embodiments, the cell volume parameter is a geometric mean cell volume of a white blood cell population. The geometric mean is similar to the arithmetic mean except instead of adding the set of values and then dividing the sum by the total count of “n” values in the set, the “n” values are multiplied and then the nth root of the resulting product is taken.

In still other embodiments, the cell volume parameter is a median (i.e., the numeric value separating the higher half of a sample, a population, or a probability distribution, from the lower half) cell volume of a white blood cell population.

The term “predetermined criterion” as used herein generally refers to a cell volume parameter (or a function of a cell volume parameter, such as a threshold or cut-off value of a parameter) obtained from a blood cell population of a plurality of control blood samples from subjects known to be deficient in vitamin B12 and/or folate. In certain embodiments, the predetermined criterion is a cell volume parameter (or a function of a cell volume parameter, such as a threshold or cut-off value of a parameter) obtained from a white blood cell population of a plurality of control blood samples from the healthy normal subjects. In certain embodiments, the predetermined criterion is a cell volume parameter (or a function of a cell volume parameter, such as a threshold or cut-off value of a parameter) obtained from a white blood cell population of a plurality of control blood samples from subjects having a disease or disorder related to deficiencies in vitamin B12 and/or folate. In certain embodiments, the predetermined criterion is a cell volume parameter (or a function of a cell volume parameter, such as a threshold or cut-off value of a parameter) obtained from a white blood cell population of one or more temporally-earlier blood sample(s) of the same test subject.

The term “predetermined criterion obtained from a white blood cell population” as used herein generally refers to a cell volume parameter (or a function of a cell volume parameter, such as a threshold or cut-off value of a parameter) obtained from a white blood cell population (and not a red blood cell population) of a plurality of control blood samples from subjects known to be deficient in vitamin B12 and/or folate.

It has been found that comparing the cell volume parameters from more than one cell type further improves sensitivity and specificity of the methods described herein. Thus, in some embodiments, susceptibility to a vitamin B12 and/or folate deficiency is determined by comparing the cell volume parameters from two or more different white blood cell subpopulations to their respective predetermined criterions. For example, in some embodiments, the methods described herein further comprise (a) comparing a cell volume parameter obtained from a cell volume distribution of a first white blood cell subpopulation comprising neutrophils to a predetermined criterion obtained from a white blood subpopulation of neutrophils from a plurality of control blood samples from subjects known to be deficient in vitamin B12 and/or folate, and (b) comparing a cell volume parameter obtained from a cell volume distribution of a second white blood cell population comprising monocytes to a predetermined criterion obtained from a white blood cell subpopulation of monocytes from a plurality of control blood samples from subjects known to be deficient in vitamin B12 and/or folate.

The classical method of screening for a vitamin B12 and/or folate deficiency in a sample (i.e., determination of mean corpuscular volume (MCV)) alone is often not enough to accurately detect a vitamin B12 and/or folate deficiency in a sample. As demonstrated herein, the combination of the MCV and the MNEV (and/or MMOV) of the sample provides a more accurate detection (i.e., fewer occurrences of false positives and false negatives) of such deficiencies. Therefore, in some embodiments, the method further comprises comparing the cell volume parameter from one or more white blood cell subpopulations (i.e., myeloid cells, leukocytes, neutrophils or monocytes; or a combination of neutrophils and monocytes) in the test sample to a predetermined criterion (or criteria) for the white blood cell subpopulation(s) and comparing the cell volume parameter from a red blood cell population (e.g., erythrocytes) in the test sample to a predetermined criterion obtained from a red blood cell population from a plurality of control blood samples from subjects known to be deficient in vitamin B12 and/or folate. A vitamin B12 and/or folate deficiency in the individual is indicated or detected when the cell volume parameter of the white blood cell population meets, approaches or exceeds the predetermined criterion obtained from the white blood cell population from a plurality of control blood samples from subjects known to be deficient in vitamin B12 and/or folate and the cell volume parameter of the red blood cell population meets, approaches or exceeds the predetermined criterion obtained from the red blood cell population from a plurality of control blood samples from subjects known to be deficient in vitamin B12 and/or folate.

In some embodiments, the cell volume parameter obtained from a red blood cell population is MCV. For example, a sample having an MCV of >100 femtoliters (fl) combined with an MNEV of >140 channels (and/or an MMOV of >169 channels) indicates that the individual is deficient in vitamin B12 and/or folate. Other levels of MCV, MNEV or MMOV may be used by one of skill in the art given the teachings herein and may depend upon the population forming the predetermined criterion as well as the individual subject(s). For example, some physiological characteristics of the subject, e.g., age, general health, weight, gender, race, and the like may result in higher or lower normal, healthy averages or abnormal, B12 and/or folate deficient averages of MCV, MMOV or MNEV. One of skill in the art may adjust the appropriate measurement cutoffs in practice of the methods described herein. The invention also provides methods of assessing treatment of a vitamin B12 and/or folate deficiency in a subject. Such methods comprise the step of comparing a cell volume parameter of test blood sample from the subject to a predetermined criterion obtained from a white blood cell population before and after treatment, wherein the cell volume parameter is obtained from a cell volume distribution of a white blood cell population in the test blood sample. Effective treatment of a vitamin B12 and/or folate deficiency is determined when the cell volume parameter is below the predetermined criterion after treatment.

In yet another embodiment, the invention provides methods of identifying a subject susceptible to a vitamin B12 and/or folate deficiency comprising comparing a cell volume parameter from a test blood sample from the subject to a predetermined criterion obtained from a white blood cell population, wherein the cell volume parameter is obtained from a cell volume distribution of a white blood cell population in the test blood sample. Susceptibility to a vitamin B12 and/or folate deficiency in the subject, is indicated when the cell volume parameter approaches the predetermined criterion. In another embodiment, latent vitamin B12 and/or folate deficiency in the subject is indicated when the cell volume parameter approaches the predetermined criterion. In some embodiments, the cell volume parameter in the test blood sample of the subject may be near but not equal to or greater than the predetermined criterion, which indicates that the subject may be on the cusp of being deficient in vitamin B12 and/or folate without any clinical manifestation. In some embodiments, the cell volume parameter in the test blood sample of the subject may be near but not equal to or greater than the predetermined criterion, which indicates that the subject may be on the cusp of being anemic and deficient in vitamin B12 and/or folate without any clinical manifestation. In other embodiments, patients with latent deficiency for vitamin B12 and/or folate deficiency may not be identified as having a vitamin B12 and/or folate deficiency because they have no anemia yet but will develop a anemia due to vitamin B12 and/or folate deficiency at a future date if not treated accordingly. In some embodiments, the method described herein will be able to detect the latent deficiencies with higher sensitivity and specificity than the existing methods, as MCV.

In other embodiments, the subject may not yet be considered as being fully deficient in vitamin B12 and/or folate (i.e., the subject has an intermediate vitamin B12 and/or folate deficiency). In some embodiments, the methods further comprise the step of determining a level of vitamin B12 and/or folate in a sample from the subject, wherein a decreased level of vitamin B12 and/or folate in the sample compared to the level of vitamin B12 and/or folate in a control sample identifies the subject as being in need of treatment for a vitamin B12 and/or folate deficiency.

In other embodiments, the invention provides methods of treating a subject for a folate deficiency comprising identifying a subject in need of treatment and administering a folate supplement to the subject. The identifying step comprises comparing a cell volume parameter from a test blood sample from the subject to a predetermined criterion obtained from a white blood cell population, wherein the cell volume parameter is obtained from a cell volume distribution of a white blood cell population in the test blood sample. Folate deficiency in the subject is indicated when the cell volume parameter meets, approaches or exceeds the predetermined criterion. In some embodiments, the identifying step optionally comprises determining the level of serum folate in a biological sample from the subject, wherein a decreased level of serum folate in the sample compared to the level of serum folate in a control sample identifies the subject as having a folate deficiency.

An exemplary method for determining the level of folic acid in a sample includes obtaining a sample (e.g., blood or plasma) from a subject and measuring the level of folate in the sample. Normal level of folic acid in adults typically is >3 ng/mL in plasma and typically is >164 ng/mL within red blood cells. An intermediate folate deficiency typically ranges between 2.5-3.0 ng/mL in plasma. By certain standards, a subject is considered deficient in folate if <2.5 ng/mL is present in the plasma of the subject. Other levels of folic acid or folate may be used by one of skill in the art given the teachings herein and may depend upon the population forming the predetermined criterion as well as the individual subject(s). For example, some physiological characteristics of the subject, e.g., age, general health, weight, gender, race, and the like may result in higher or lower normal, healthy averages or abnormal, B12 and/or folate deficient averages of folate or folic acid. One of skill in the art may adjust the appropriate measurement cutoffs in practice of the methods described herein.

In yet another embodiment, the invention provides methods of treating a subject for a vitamin B12 deficiency comprising identifying a subject in need of treatment and administering a vitamin B12 supplement to the subject. The identifying step comprises comparing a cell volume parameter from a test blood sample from the subject to a predetermined criterion obtained from a white blood cell population, wherein the cell volume parameter is obtained from a cell volume distribution of a white blood cell population in the test blood sample. Vitamin B12 deficiency in the subject is indicated when the cell volume parameter meets, approaches or exceeds the predetermined criterion. In some embodiments, the identifying step optionally comprises determining a level of vitamin B12 in a biological sample from the subject, wherein a decreased level of vitamin B12 in the sample compared to the level of serum folate in a control sample identifies the subject as having a folate deficiency.

In certain embodiments, any of the methods described herein or portions thereof are performed by a computer processor or computer-programmed instrument that generates numerical or graphical data useful in detecting the deficiency, with or without anemia or monitoring of the treatment. For example, measuring functions may be performed by a computer processor and/or comparing processes may be performed by the computer processor. Either process may lead to generation of numerical or graphical data for interpretation by the physician.

In any of the methods described herein, the diagnosis, detection or prognostic determination can include integrating the comparative relationship of the white cell volume parameter of test sample with the predetermined criterion with the presentation of clinical symptoms of the B12 and/or folate deficiency in the subject.

In any of the methods described herein, the diagnosis, detection or prognostic determination can include integrating or coupling the comparative relationship of the white cell volume parameter of test sample with the predetermined criterion with familial history of B12 and/or folate deficiency.

In still other embodiments, these methods permit determination of a quantitative assessment of the likelihood or risk of B12 deficiency and/or folate deficiency occurrence in a subject that has not yet developed clinical symptoms of the deficiency.

Exemplary methods for determining the level of vitamin B12 in a biological sample (e.g., blood) include measuring the concentration of holotranscobalamin (the active component of vitamin B12) in the sample (Clarke et al., Clin. Chem., 53:963-970, 2007); or by measuring the level of vitamin B12 in the sample. Normal blood levels of vitamin B12 typically ranges between 180-914 pg/mL. An intermediate vitamin B12 deficiency typically ranges between 146-180 pg/mL. By certain standards a subject is considered deficient in vitamin B12 if the blood sample has =145 pg/ml of vitamin B12. It is known in the art that as the vitamin B12 level decreases, the level of methylmalonic acid in the blood increases. Thus, a methylmalonic acid (MMA) blood test can be performed to aid evaluation of vitamin B12 test results (Oh et al., American Family Physician, 67:979-986 and 993-994, 2003.

It has been found that the properties of white blood cells of a subject deficient in vitamin B12 and/or folate are altered. Particularly the volumes of the specific white blood cell subpopulations are altered. More specifically, it has been found that when measured by the VCS measurement, the DC impedance means of monocytes and neutrophils increase to levels that the differences of these parameters from those of normal blood samples are statistically significant. Alterations of these parameters of white blood cell subpopulations can be utilized as an indicator for susceptibility of a subject for a vitamin B12 and/or folate deficiency.

It should be understood that in certain embodiments, increases in the mean measurement of DC impedance of the monocytes are caused by the presence of large monocytic cells (also called megaloblastic monocytes) that result due to vitamin B12 and or folate deficiency.

In some embodiments, and as demonstrated in the Example provided herein, the cell volume and its distribution of one or more white blood subpopulations (and optionally in combination with a red blood cell population) can be determined by the DC impedance measurement. It should be understood, however, the cell volume can also be determined by other suitable means as described herein, including forward or low angle light scatter, or axial light loss measurement. In general, the low angle light scatter is defined as the light scatter signals measured in less than 10° from the incident light. Axial light loss ((ALL), also known as forward extinction) is generally the decrease in light energy due to a particle passing through a beam of incident light and being detected by a photo-detector. Generally ALL signals are detected at an angle from about 0° to about 2° from the incident light. Both low angle light scatter and ALL signals are strongly influenced by the size of the cell. The apparatus and method of measuring low angle light scatter and ALL signals is known in the art and, in some embodiments, is used for measuring cell volume.

In some embodiments, laser rastering is used to determine the cell volume of an individual white blood cell population (and/or red blood cell population). See, U.S. Patent Publication Nos. 2008/0158561.

Any commercial hematology analyzer capable of analyzing a white blood cell population (optionally in combination with a red blood cell population) is used to practice the methods described herein. In one embodiment, the commercial hematology analyzer is the Coulter® LH750 or the Coulter® GEN*S™ hematology analyzer (Beckman Coulter, Inc. Brea, Calif.). On both of these analyzers, several aliquots of a blood sample are analyzed concurrently in different analysis modes. In the complete blood count (CBC) mode, a first aliquot of a blood sample is diluted with an isotonic blood diluent to form a first sample mixture, and red blood cells are analyzed from the first sample mixture. At the same time, a second aliquot of the blood sample is mixed with a blood diluent and a lytic reagent to form a second sample mixture. Hemoglobin levels and white blood cells are analyzed from this second sample mixture.

In some embodiments, such measurements provided by the commercial hematology analyzers include, but are not limited to, MCV of red blood cells (e.g., erythrocytes), percentage of reticulocytes in the sample, number of reticulocytes in the sample, white blood cell population data (including, but not limited to, MNEV and MMOV), serum iron level, serum ferritin level, level of vitamin B12, serum folate level, red blood cell folate, serum erythropoietin level, transferrin level, C-reactive protein level, and level of intrinsic factor antibody.

In one aspect, the methods described herein comprise combining the whole blood sample with a lytic reagent system. This step can occur before further measurement or comparison with controls. In such embodiments, the lytic reagent system is used to lyse red blood cells and to preserve the integrity of the white blood cells in the sample. In some embodiments, the lytic reagent system comprises a lytic reagent and a stabilizing reagent. Exemplary lytic reagents, stabilizing reagents and the method of use have been described in U.S. Pat. Nos. 5,155,044; 5,731,206; 5,786,224; 5,686,308, 5,843,608; 6,573,102 and 6,869,798. Further exemplary lytic and stabilizing reagents include Lyse S® III Diff lytic reagent and Isoton® III diluent, Erythrolyse™, Stabilyse™ and manufactured by Beckman Coulter, Inc., California. Alternatively, the reagent system can also be an isotonic lysing reagent as described in U.S. Pat. No. 5,882,934. This reagent dilutes the blood sample and lyses the red blood cells at the same time. It should be understood that other lytic reagents known in the art to be suitable to preserve white blood cells during the measurement of the sample mixtures can also be used for the purpose of the present invention.

Using the lytic reagents described above, the white blood cells are in some instances partially lysed. The obtained cell distribution depends on, in a certain degree, the nuclear volumes of white blood cell subpopulations. Typically, from this cell distribution the white blood cells can be differentiated into two or three subpopulations, commonly referred to as a two-part differential or a three-part differential. The three-part differential differentiates the white blood cells into lymphocytes, monocytes and granulocytes. In some embodiments, such cell distribution characteristics are utilized to further improve sensitivity and specificity of susceptibility to a vitamin B12 and/or folate deficiency using the method described above which is based on the analysis of the first sample mixture.

On the commercial hematology analyzer using the VCS measurement, a three-dimensional scattergram is produced. The three dimensions are DC impedance as the z-axis, which is also referred to as “V” because a DC impedance signal directly correlates to the volume or size of a cell; Opacity (OP) as the y-axis, which is a function of RF impedance and DC impedance, and also referred to as “C” or conductivity because the RF impedance signals further reflect cell contents of a cell; and Rotated Light Scatter (RLS) as the x-axis, which is a function of light scatter and DC impedance, and also abbreviated as “S”. The three-dimensional scattergram is usually displayed as a DC impedance vs. RLS two dimensional scattergram, or as a DC impedance vs. OP two dimensional scattergram. In the DC impedance vs. RLS scattergram, four major white blood cell subpopulations, i.e., lymphocytes, monocytes, neutrophils, and eosinophils, are differentiated from one another. In the DC impedance vs. OP scattergram, three major groups of white blood cell subpopulations are differentiated from one another, i.e., monocytes and a sum of neutrophils and eosinophils. The basophils can be differentiated using one or more gated scattergram. It order to differentiate monocytes, or neutrophils from other cell types, all three measurements can be used, i.e., DC impedance, RF impedance and RLS, or only two measurements, either DC impedance and LS, or DC impedance and RF impedance. Furthermore, various other methods such as multi-angle light scatter measurement using forward scatter and side scatter, or low angle and medium angle light scatter can be used for differentiating white blood cell subpopulations, particularly monocytes, or neutrophils from other cell types.

In the methods described herein, the test subject and control subjects are mammalian. By mammalian subjects is meant primarily humans, but can also mean other mammals including domestic animals including animals of value as pets, laboratory specimens, and the like.

The following Example is illustrative of the invention and is in no way to be interpreted as limiting the scope of the invention, as defined in the claims. It will be understood that other variations of the methods may be employed, in accordance with the proceeding disclosure.

### Example 1

### Detection of Vitamin B12 and/or Folate Deficiency by Analyzing a White Blood Cell Population in a Sample

The following Example demonstrates the frequency of a situation in which a patient has a normal MCV while having a vitamin B12 and/or folate deficiency (without anemia). This Example also demonstrates that in embodiments of the methods described herein white cell volume parameters alone or multiple parameters (e.g., of both red blood cell and white blood cell populations) can be evaluated to accurately detect vitamin B12 and/or folate deficiencies in a sample. One hundred eight cases of anemia defined according to the World Health Organization (WHO) anemia criteria (men=hemoglobin (hb)<13 g/dL and women=hb<12 g/dL) were collected from hospital laboratory samples and samples were randomly selected from those collected. Blood samples from fifty-eight individuals considered as being normal, healthy individuals were used as a control. The WHO criteria of anemia were the only condition considered.

The study population included 82 males and 84 females. All samples were analyzed using a commercial hematology analyzer (LH 700 Series analyzer and associated LH reagents, Beckman Coulter). The hematology analyzer provided two separate white blood cell differential analyses. The first differential analysis was performed using the VCS measurement of the first sample mixture prepared by mixing a first aliquot blood sample with an amount of Erythrolyse™ II to lyse red blood cells and subsequently mixing with an amount of Stabilyse™ to stabilize the white blood cells. The VCS measurement differentiated the white blood cells into five subpopulations, i.e., lymphocytes, monocytes, neutrophils, basophils and eosinophils. The second differential analysis was performed by using a DC impedance measurement of the second sample mixture prepared by diluting a second aliquot blood sample with Isoton® III diluent and mixing with an amount of Lyse S® III Diff reagent. The DC impedance measurement provided a total count of white blood cells and also differentiated the white blood cells into three subpopulations, i.e., lymphocytes, monocytes and granulocytes using a one dimensional DC impedance histogram, which is commonly referred to as a WBC histogram. For the purpose of reflecting the detection method, it is herein referred to as a WBC DC histogram. All reagents described above are manufactured by Beckman Coulter, Inc., California.

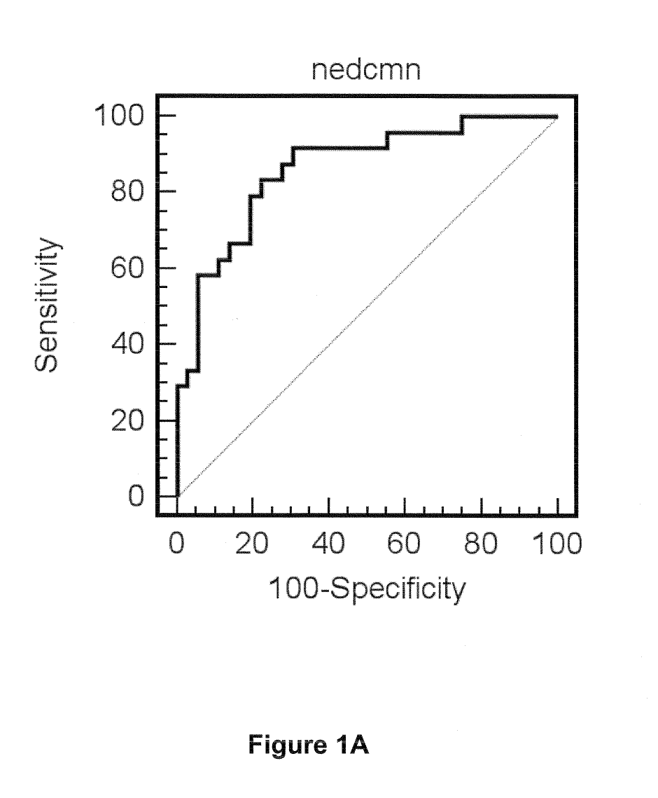

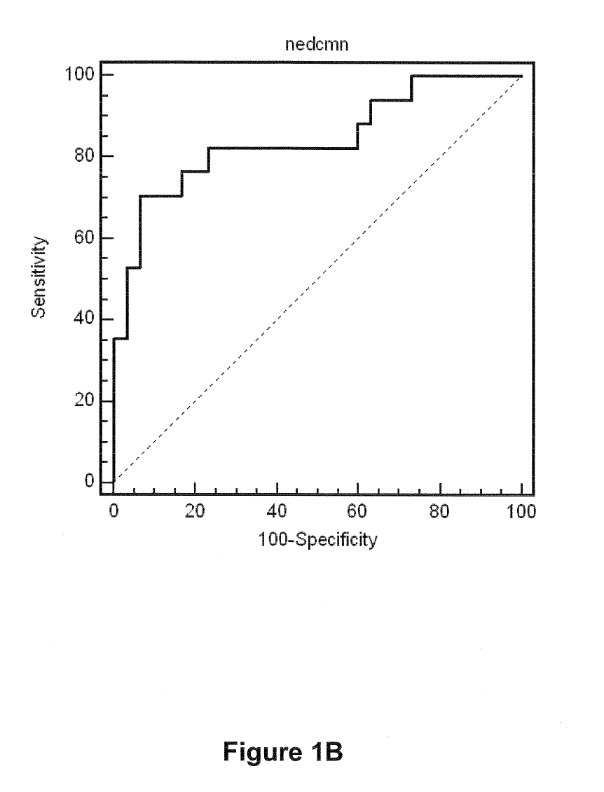

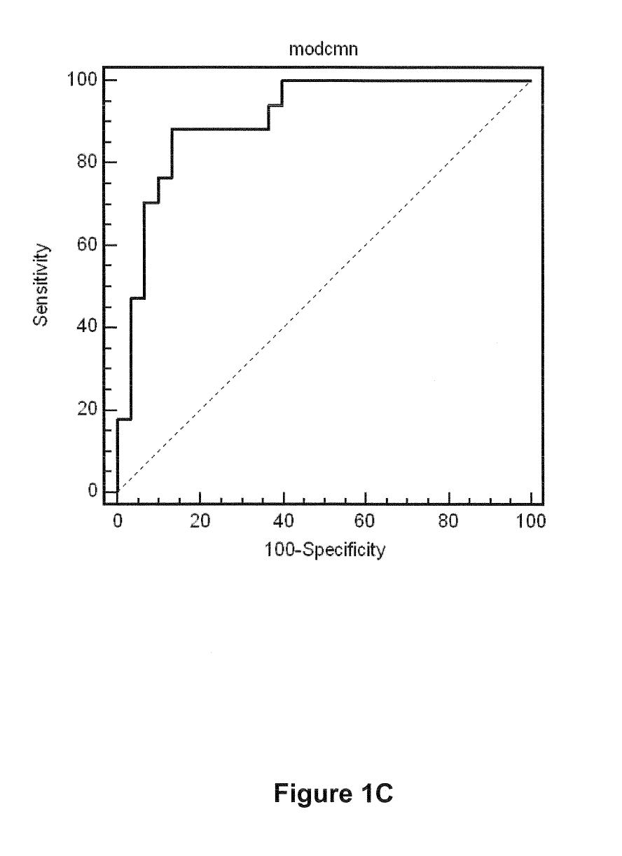

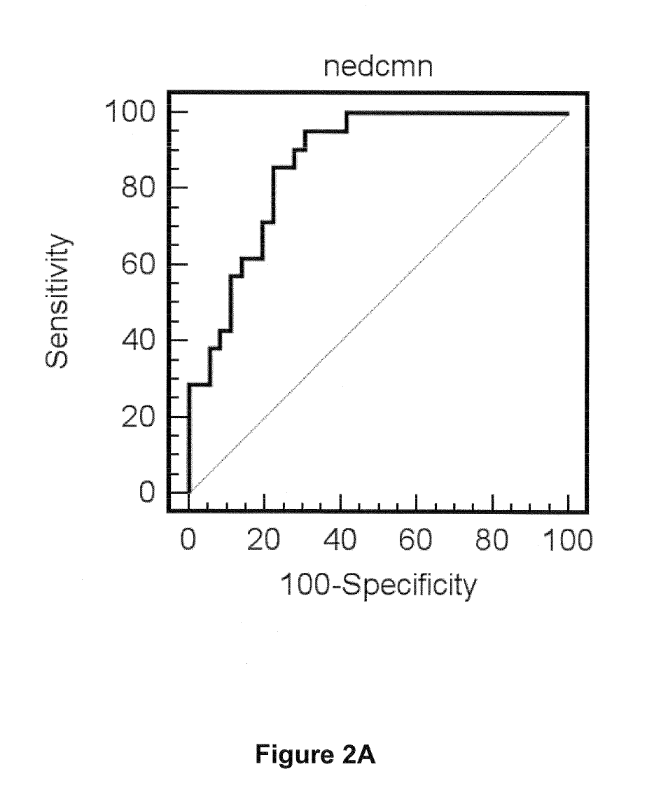

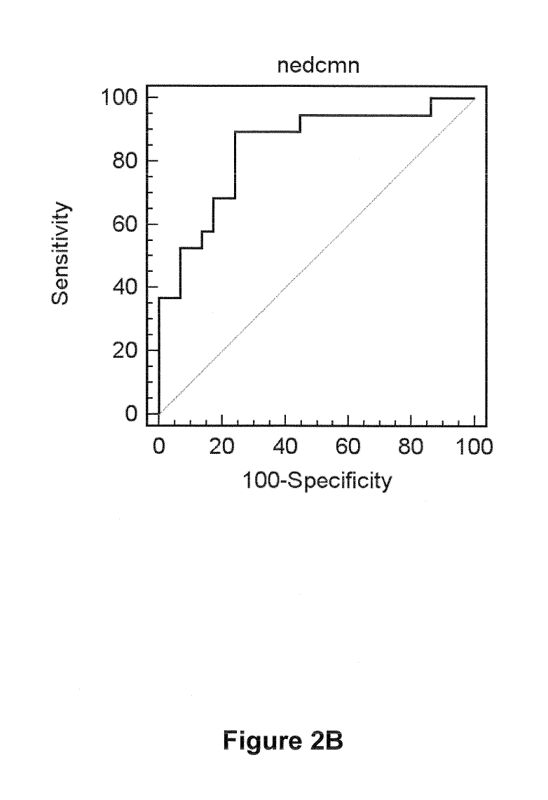

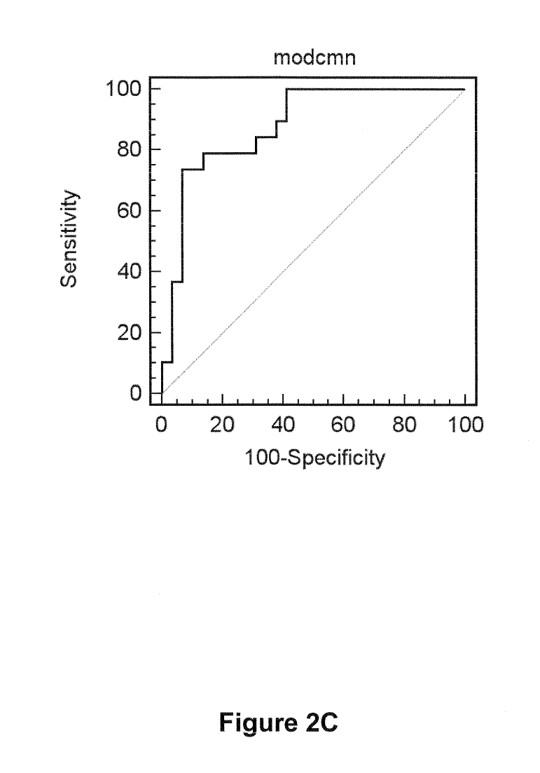

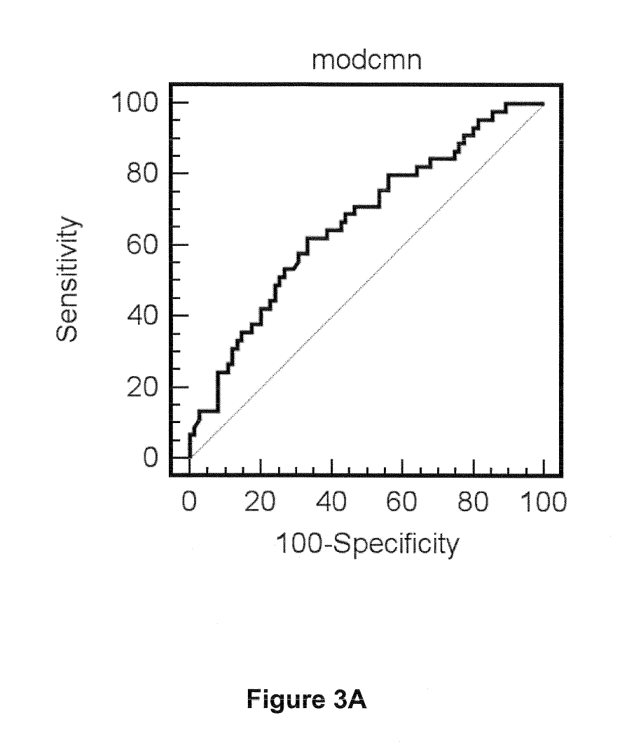

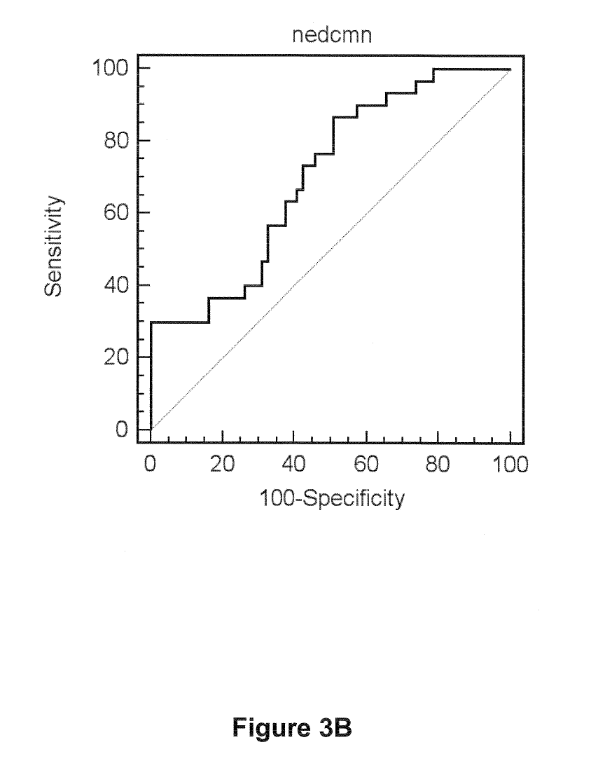

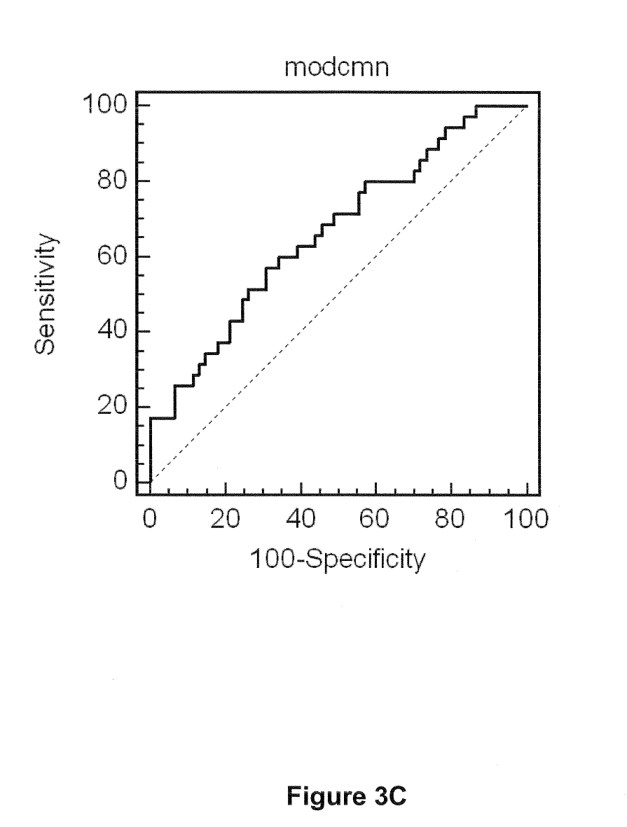

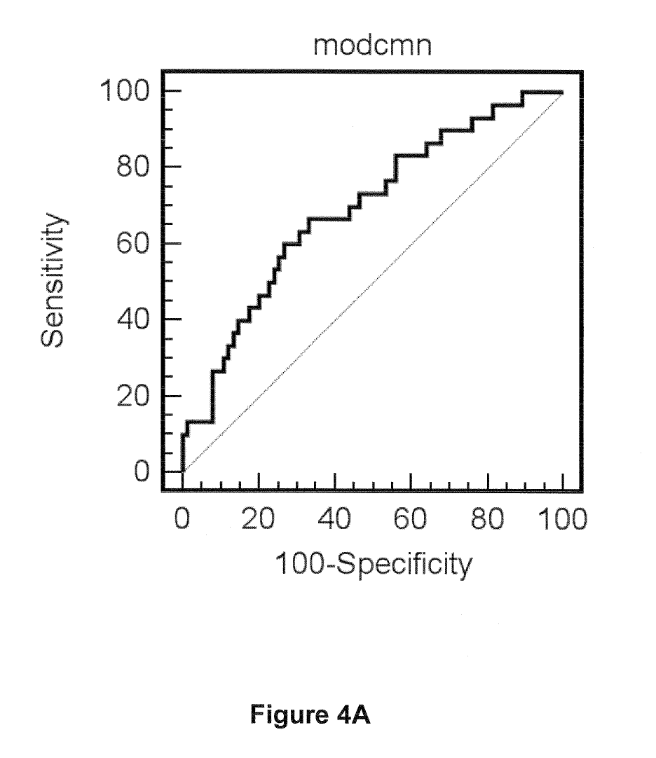

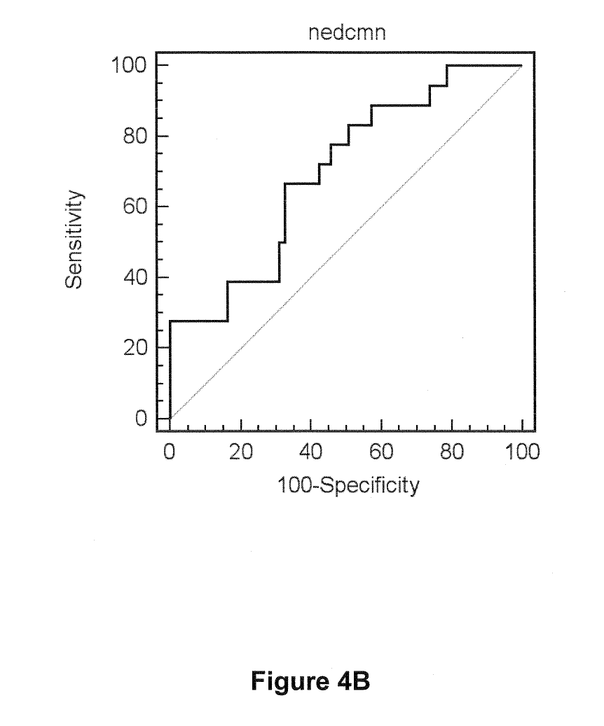

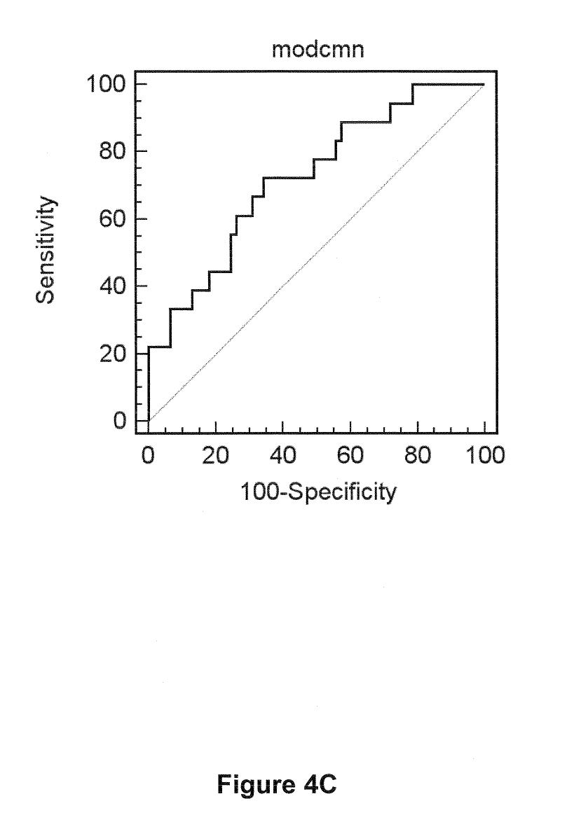

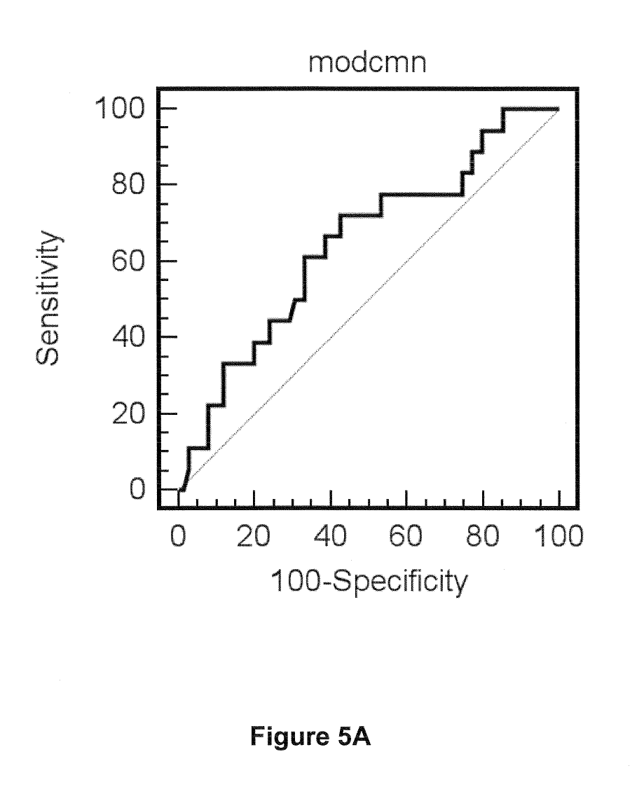

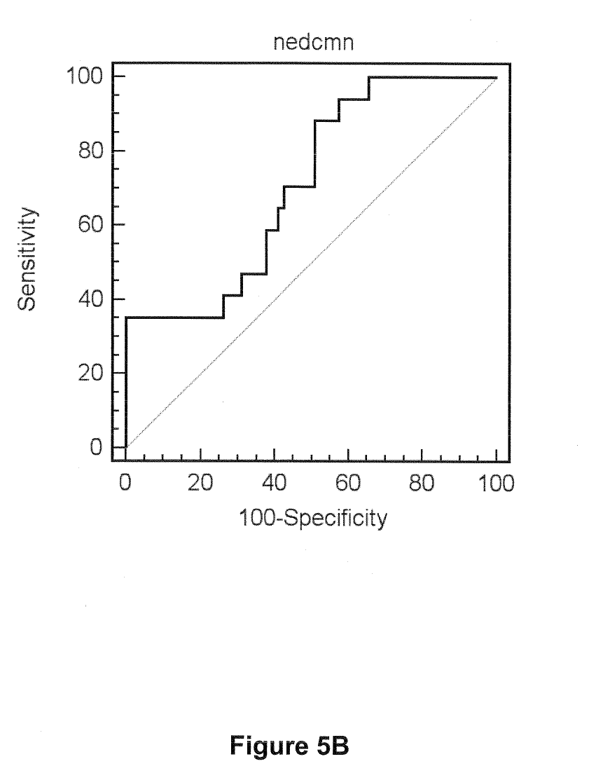

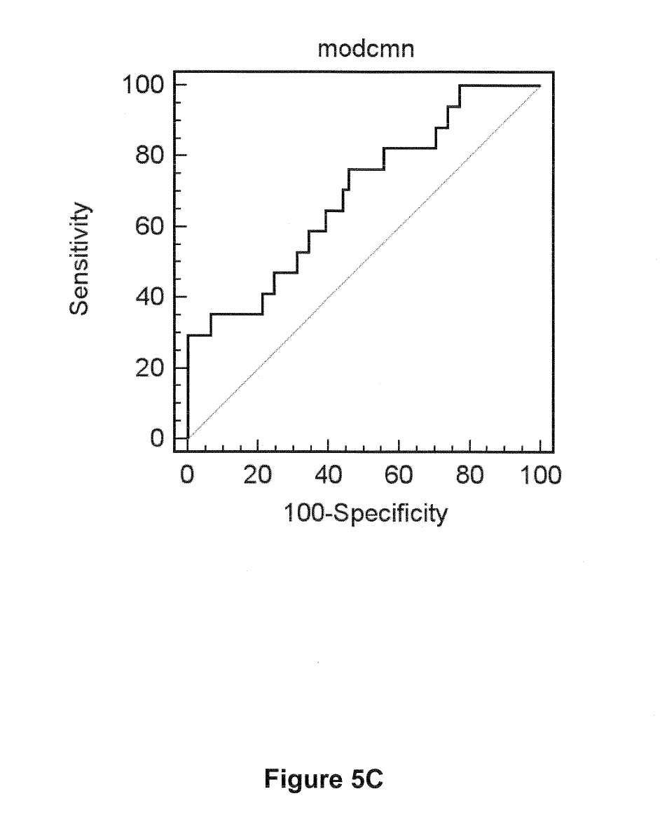

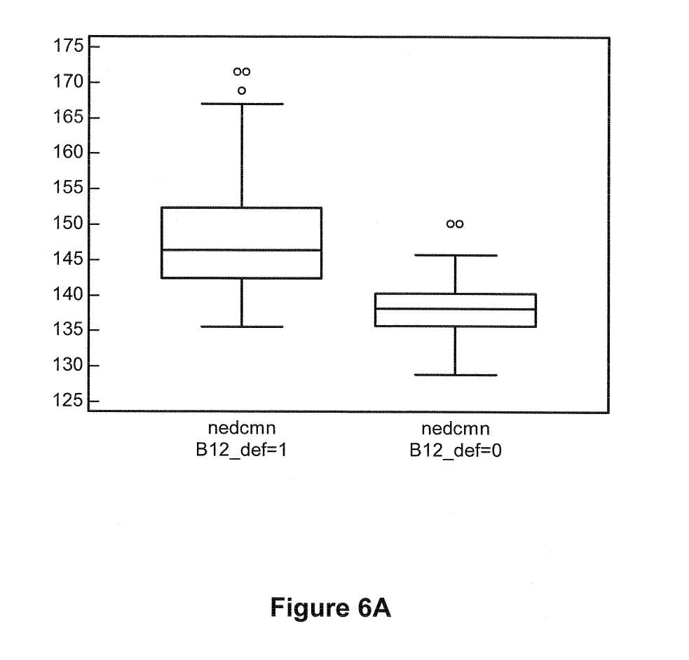

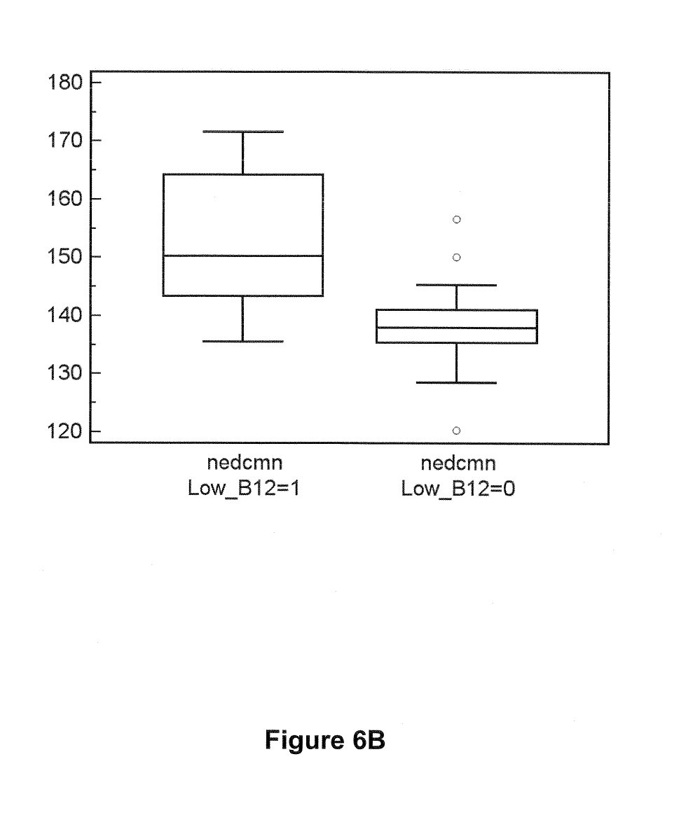

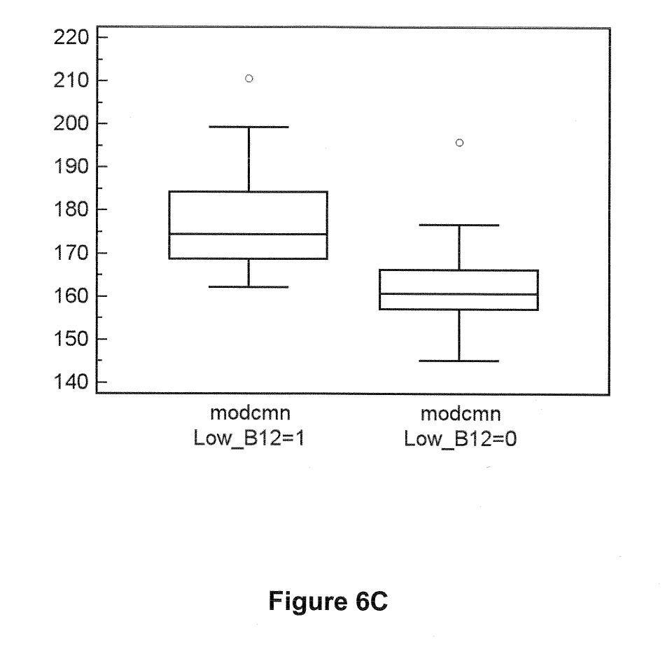

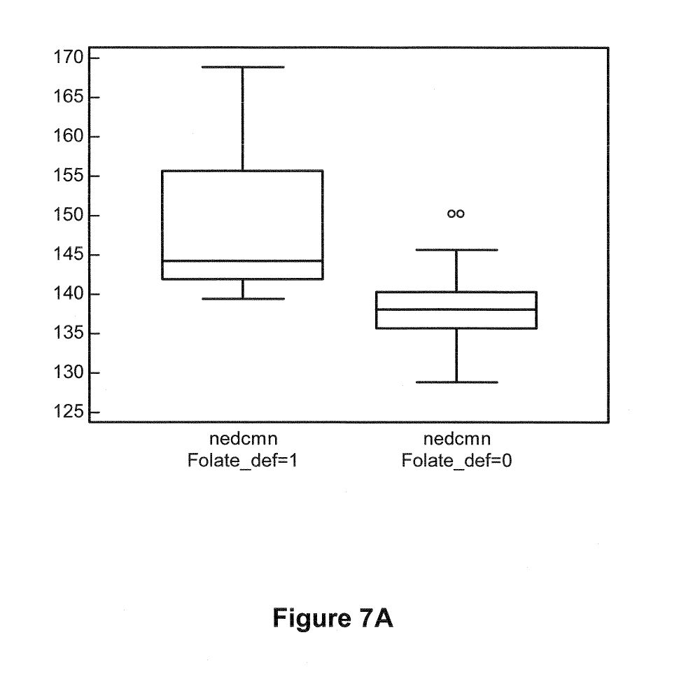

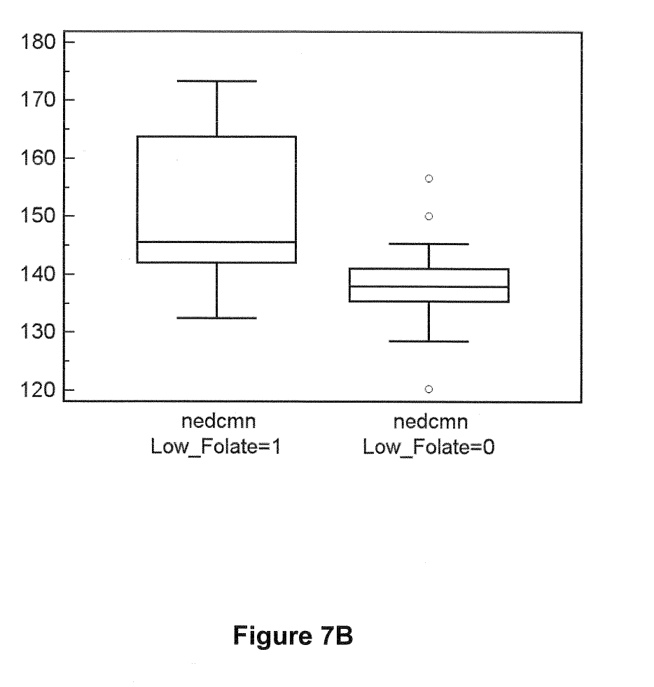

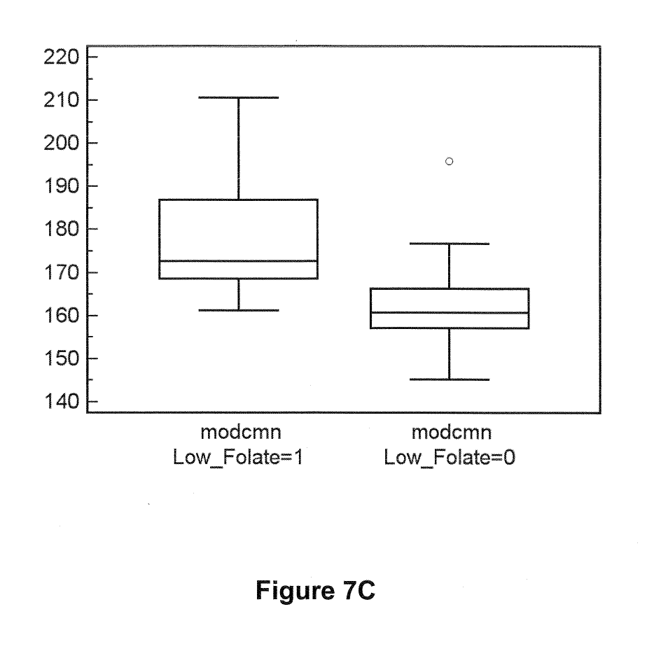

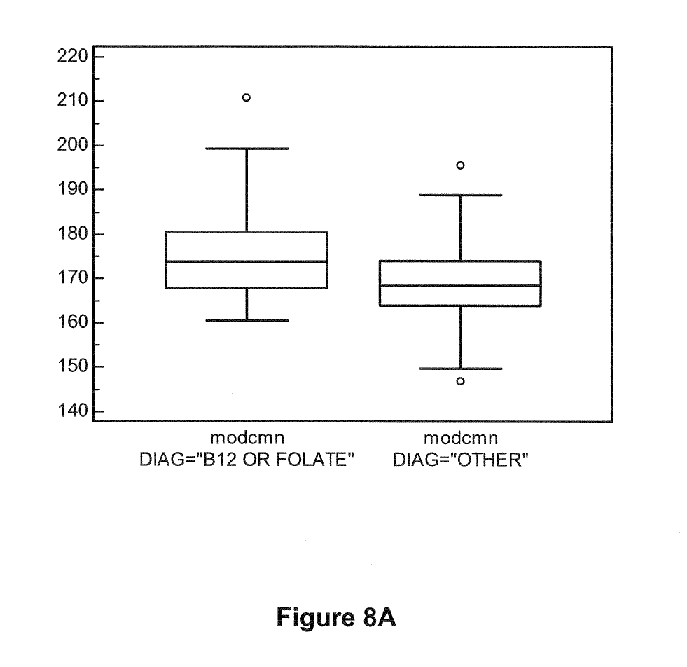

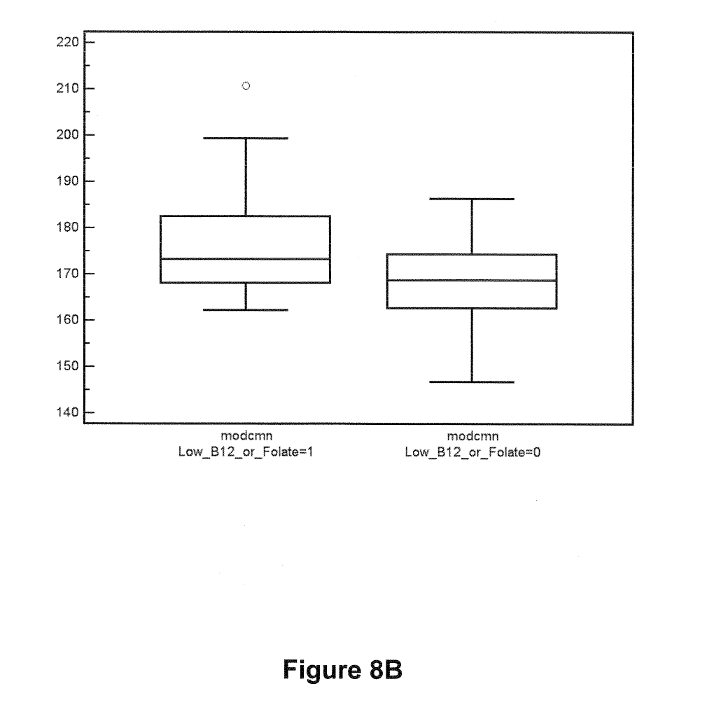

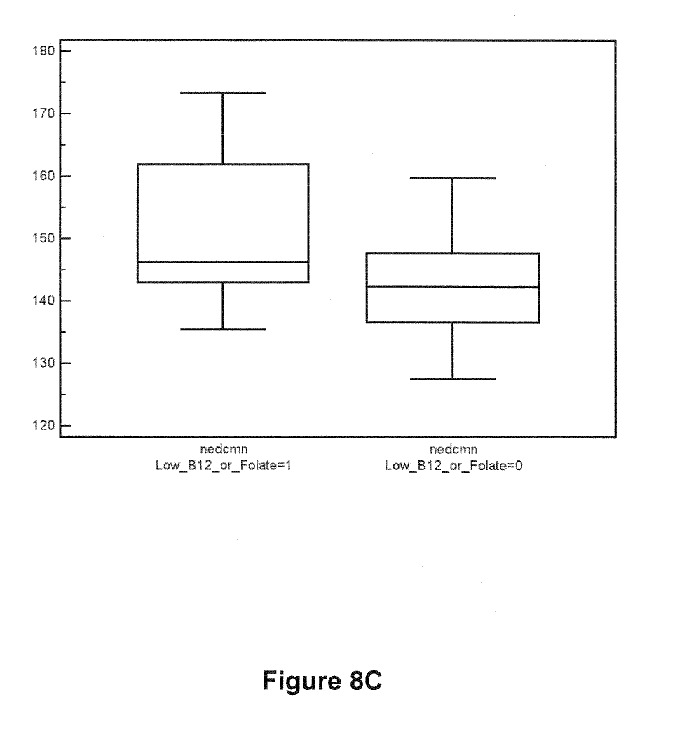

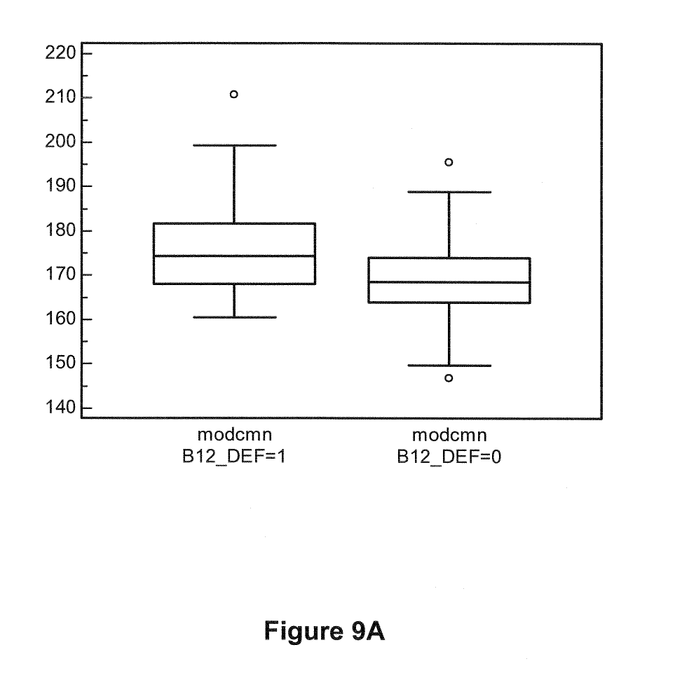

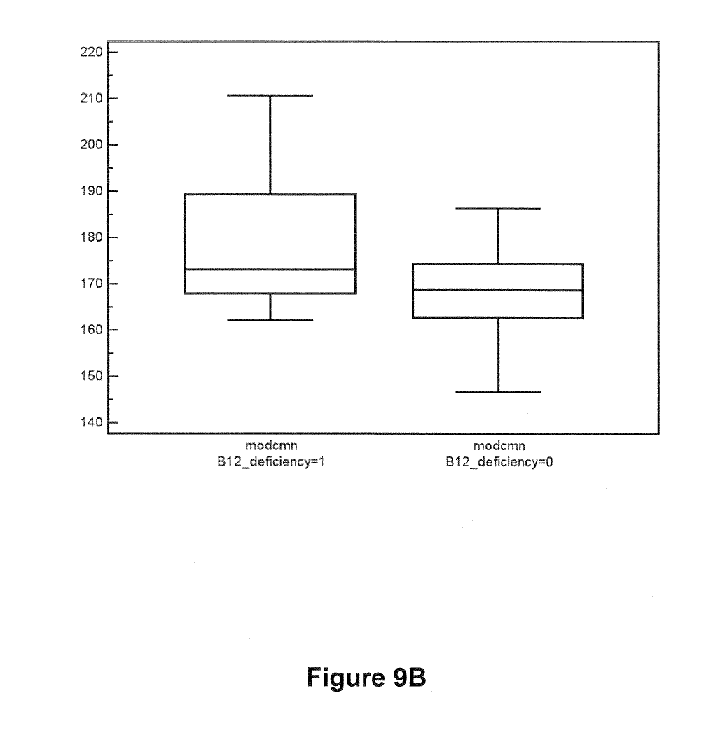

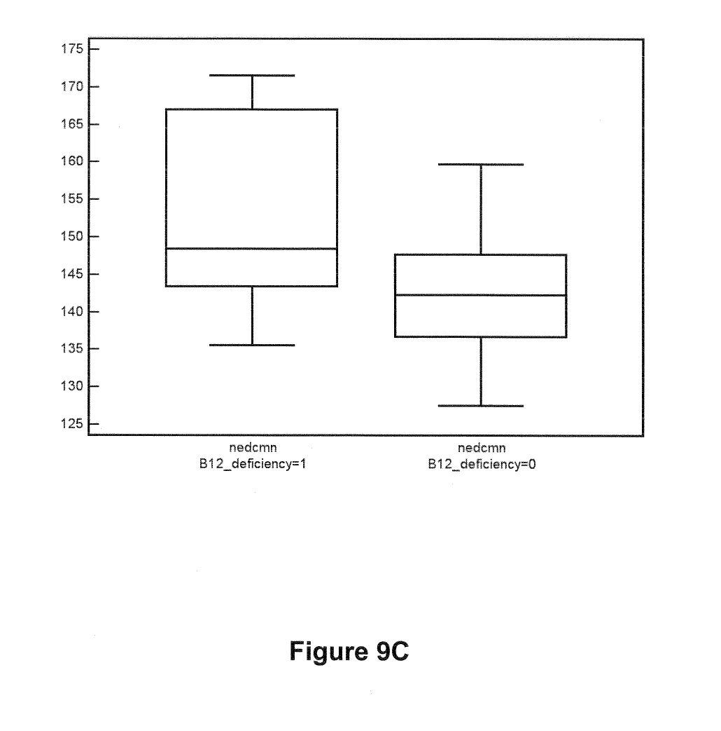

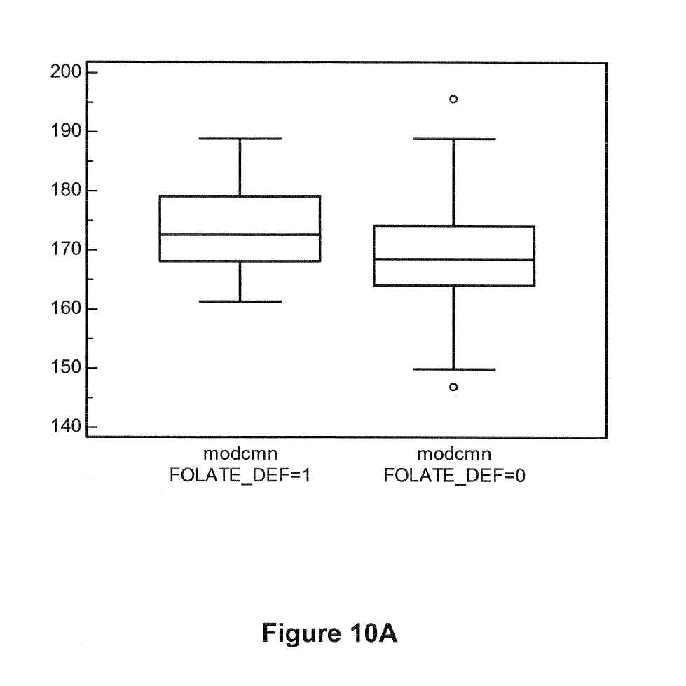

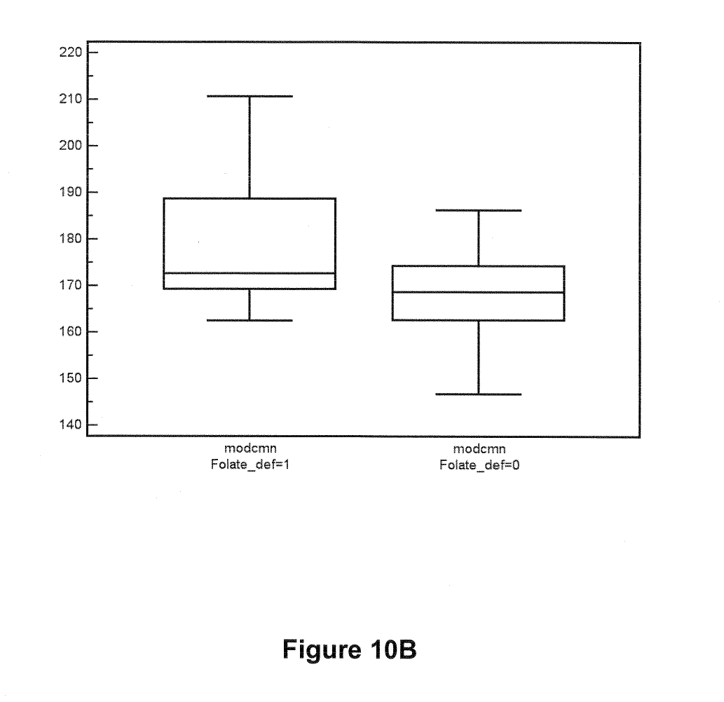

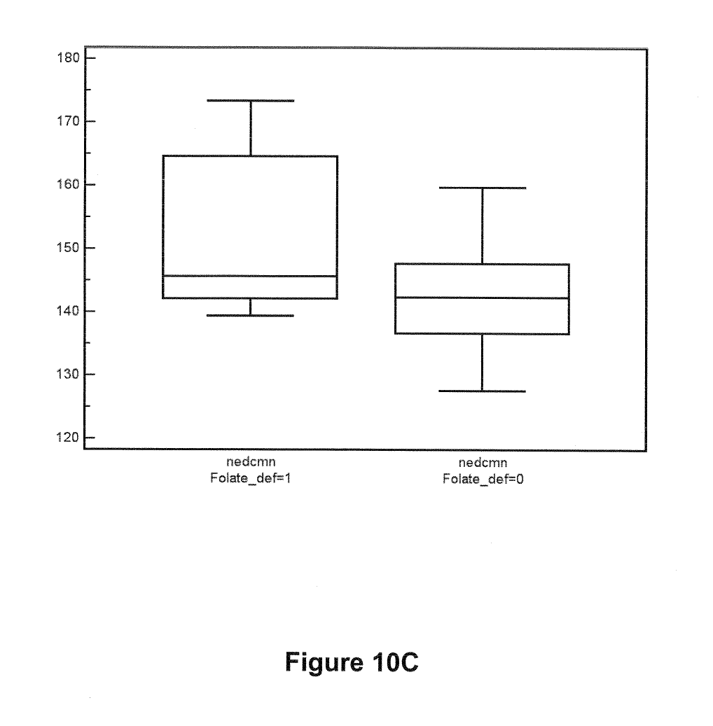

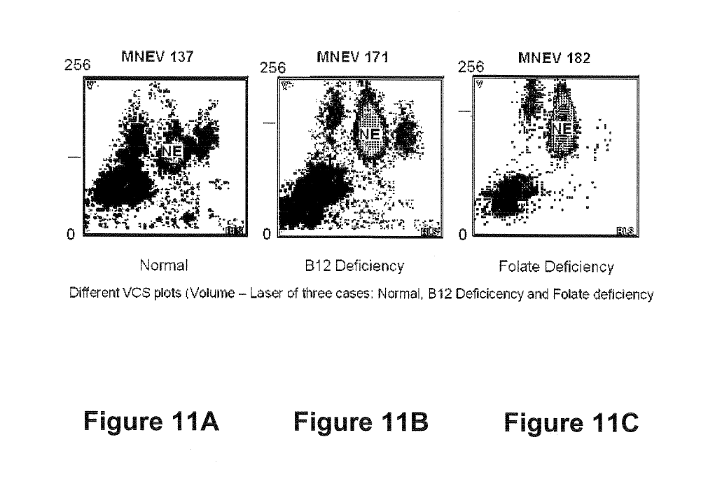

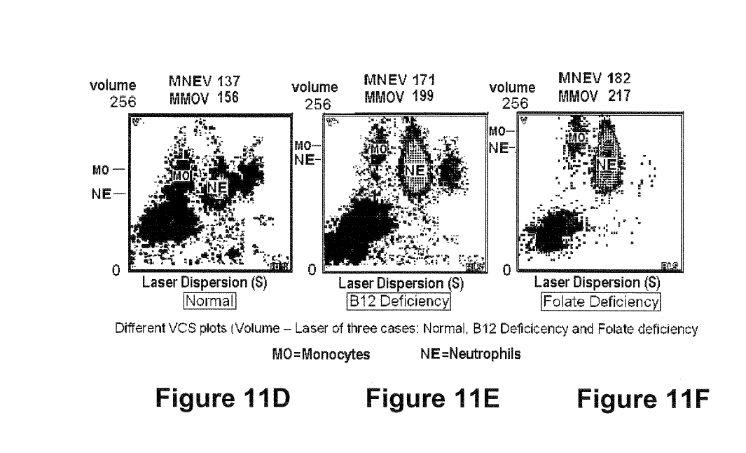

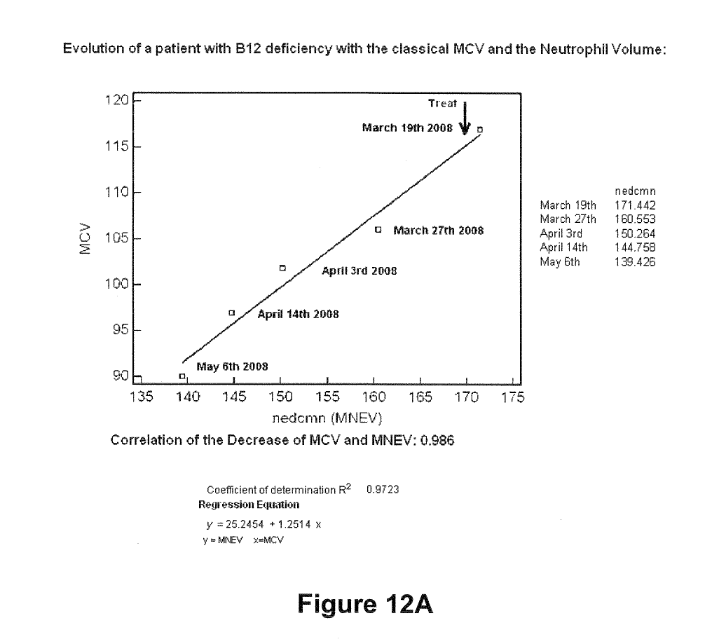

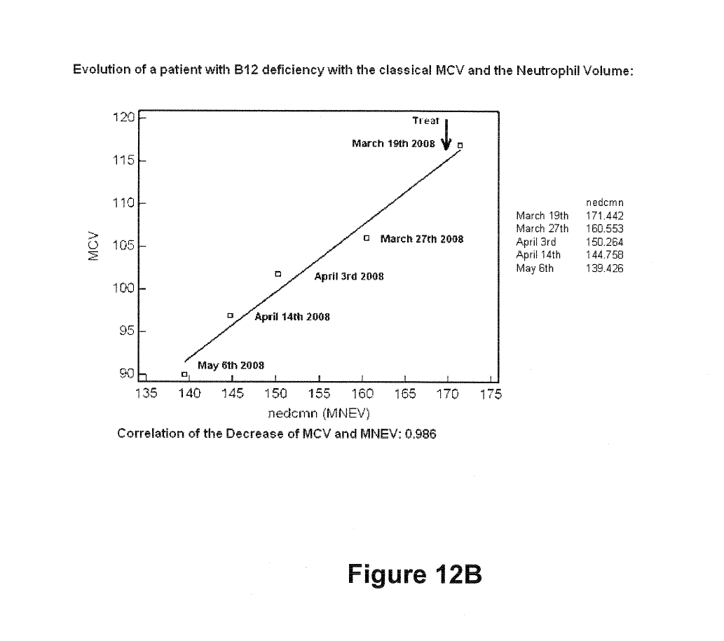

The following parameters of the samples were analyzed: Complete blood count (CBC) with differential (CBC-Diff, i.e., provides cell count of neutrophils, lymphocytes, monocytes, eosinophils and basophils), percentage of reticulocytes in the sample, number of reticulocytes in the sample, white blood cell population data (e.g., number and cell volume of neutrophils, monocytes, granulocytes, basophils), serum iron level, serum ferritin level, level of vitamin B12, serum folate level, red blood cell folate, serum erythropoietin level, transferrin level, C-reactive protein level, and level of intrinsic factor antibody. The efficiency of the classical hematological parameters (i.e., MCV) compared to the efficiency of white blood cell parameters (i.e., MNEV or MMOV) was also analyzed. Finally, receiver operator characteristic (ROC) curves were plotted and the corresponding area under the curve (AUC) was calculated to assess their potential clinical utility (See FIGS. 1A, 2A, 3A, 4A and 5A and Table 2 below). The ROC curve plots sensitivity versus 100-specificity, wherein the greater deviation from the x,y identity line indicated a greater diagnostic utility. The area under the curve is indicative of the use of the MNEV (or MMOV) to distinguish subjects deficient in vitamin B12 and/or folate from normal subjects or subjects with anemias not caused by the vitamin B12 and/or folate deficiency.

Statistical Analyses: Data were analyzed and statistical significance was determined using the Mann-Whitney U test and Student's t-test, as appropriate. The Receiver Operating Characteristic (ROC) curves were determined using MedCalc software (MedCalc Software, Mariakerke, Belgium).

Table 1 provides descriptive statistics for the different groups of patients and normal subjects. The values are the mean of the parameters for the group of n patients.

TABLE 1
Descriptive Statistics

| | | Mean values different groups of patients | | | | |
|---|---|---|---|---|---|---|
| | | n | MCV | NE Mean Volume | MO Mean Volume | |
| non anemic B12 | | | | | | B12 |
| Hb > 12 | low B12 | 3 | 105.9 | 150.3 | 180.0 | 90.0 |
| Hb > 12 | Interm B12 | 3 | 87.5 | 141.5 | 170.3 | 168.7 |
| Hb > 12 | normal B12 | 52 | 91.4 | 138.9 | 163.2 | 434.1 |
| Folate | | | | | | Folate |
| Hb > 12 | low Folate | 2 | 91.2 | 137.3 | 167.8 | 1.93 |
| Hb > 12 | normal Folate | 56 | 92.0 | 139.7 | 164.3 | 5.65 |
| anemic B12 | | | | | | B12 |
| Hb < 12 | low B12 | 21 | 92.06 | 150.08 | 176.56 | 97.8 |
| Hb < 12 | Interm B12 | 3 | 86.53 | 144.75 | 179.02 | 160.3 |
| Hb < 12 | normal B12 | 84 | 87.22 | 142.58 | 167.46 | 593.7 |
| Folate | | | | | | Folate |
| Hb < 12 | low Folate | | 87.95 | 147.197 | 171.88 | 1.626 |
| Hb < 12 | normal Folate | | 88.16 | 143.78 | 169.31 | 6.02 |
| B12-Folate | | | | | | |
| Hb > 12 | Normal Folate B12 | 51 | 91.4 | 139 | 163.3 | |
| Hb < 12 | Low Folate and B12 | 32 | 90 | 149.1 | 175.3 | |
| Other anemias | Other anemias | 76 | 87.3 | 142 | 167.1 | |

Table 2 shows the comparative statistics between the different groups of normals and patients with or without Folate or B12 deficiencies. As shown above, the cut-off values obtained from the ROC analysis are used as the criterion for indication of a vitamin B12 and/or folate deficiency.

Comparative Statistics

| | | | | | | |
|---|---|---|---|---|---|---|
| Mann Withney | T-Test | ROC AUC | Sens | Specif | Cut-off | significance ROC |

B12 def vs Normals

| | | | | | | | |
|---|---|---|---|---|---|---|---|
| MCV | p = 0.3156 | p = 0.9533 | 0.577 | 45.8 | 91.7 | | p = 0.3037 |
| NE Mean Volume* | p < 0.0001 | p < 0.0001 | 0.862 | 83.3 | 77.8 | >140.32 | p = 0.0001 |
| MO Mean Volume | p < 0.0001 | p < 0.0001 | 0.829 | 62.5 | 88.9 | >172.32 | p = 0.0001 |

Folate def vs Normals

| | | | | | | | |
|---|---|---|---|---|---|---|---|
| MCV | p = 0.0798 | p = 0.6461 | 0.665 | 66.7 | 75 | <89.5 | 0.0229 |
| NE Mean Volume* | p = 0.0008 | p < 0.0001 | 0.87 | 95.2 | 69.4 | >140.16 | p = 0.0001 |
| MO Mean Volume | p = 0.0002 | p = 0.0002 | 0.801 | 66.7 | 83.3 | >169.58 | p = 0.0001 |

B12 or Folate deficiency vs Other anemias

| | | | | | | | |
|---|---|---|---|---|---|---|---|
| MCV | p = 0.1783 | p = 0.0658 | 0.573 | 26.7 | 93.4 | >99.7 | p = 0.1779 |
| NE Mean Volume | p = 0.0440 | p = 0.0132 | 0.61 | 77.8 | 48 | >141.336 | p = 0.0415 |
| MO Mean Volume* | p = 0.0024 | p = 0.0009 | 0.666 | 62.2 | 66.7 | >172.35 | p = 0.0015 |

B12 deficiency vs Other anemias

| | | | | | | | |
|---|---|---|---|---|---|---|---|
| MCV | p = 0.2006 | p = 0.0723 | 0.58 | 33.3 | 93.4 | >99.7 | p = 0.2037 |
| NE Mean Volume | p = 0.0631 | p = 0.0208 | 0.616 | 76.7 | 48 | >141.336 | p = 0.0628 |
| MO Mean Volume* | p = 0.0020 | p = 0.0006 | 0.694 | 66.7 | 66.7 | >172.35 | p = 0.0012 |

Folate deficiency vs Other anemias

| | | | | | | | |
|---|---|---|---|---|---|---|---|
| MCV | p = 0.1495 | p = 0.0939 | 0.61 | 83.3 | 38.2 | >85 | p = 0.1550 |
| NE Mean Volume | p = 0.1501 | p = 0.0734 | 0.61 | 88.9 | 42.7 | >140.903 | p = 0.1550 |
| MO Mean Volume* | p = 0.0554 | p = 0.0498 | 0.646 | 72.2 | 57.3 | >170.02 | p = 0.0562 |

Results indicated that 5.2% of the “normal” patients (i.e., patients having a normal MCV) with a normal hemoglobin level had a vitamin B12 deficiency (vitamin B12<145 pg/mL); 5.2% of the “normal” patients (i.e., patients having a normal MCV) with a normal hemoglobin level had intermediate values of vitamin B12 (vitamin B12>145<180 pg/mL); and 3.5% of the “normal” patients (i.e., patients having a normal MCV) with a normal hemoglobin level had low serum folate.

From the group of anemic patients, results indicated that the prevalence of vitamin B12 deficiency was 21/108 (19.4%), the prevalence of intermediate vitamin B12 levels was 3/108 (2.8%), the presence of serum folate deficiency was 10/108 (9.3%), the prevalence of vitamin B12 and serum folate deficiency was 1/108 (0.9%), and the prevalence of intermediate vitamin B12 levels and serum folate deficiency was 1/108 (0.9%).

Results indicated that after comparing the hematological tests of control individuals with the tests of patients with a vitamin B12 deficiency, MNEV was more accurate in detecting samples with the B12 deficiency followed by the MMOV. When comparing patients with vitamin B12 or folate deficiencies with patients with anemia (without such deficiencies), the most accurate test was the MMOV followed by the MNEV. MMOV was determined to be the best parameter for detecting latent vitamin B12 deficiency in a sample.

### Example 2

### Detection of Vitamin B12 and/or Folate Deficiency by Analyzing a White Blood Cell Population in a Sample

The original data presented in Example 1, was reviewed for statistical corrections at a later time. Certain changes were made in grouping and analysis of data based on more strict selection of patients for each group. All patients being treated were excluded from normals and from group with other anemias. The following Example demonstrates the frequency of a situation in which a patient has a normal MCV while having a vitamin B12 and/or folate deficiency (without anemia). This Example also demonstrates that multiple parameters (red blood cell v. white blood cell) should be evaluated in order to accurately detect vitamin B12 and/or folate deficiencies in a sample.

One hundred nine cases of anemia defined according to the World Health Organization (WHO) anemia criteria (men=hemoglobin (hb)<13 g/dL and women=hb<12 g/dL) were collected from routine hospital blood samples. Anemia, as defined by the WHO criteria, was the only condition considered. Blood samples from fifty-seven individuals considered as being normal, healthy individuals were used as a control. The study population included 82 males and 84 females All samples were analyzed using a commercial hematology analyzer (LH 700 Series analyzer and associated LH reagents, Beckman Coulter) and the following parameters of the samples were obtained on LH700 Series analyser: CBC with differential (CBC-Diff, i.e., provides cell count of neutrophils, lymphocytes, monocytes, eosinophils and basophils), percentage of reticulocytes in the sample, number of reticulocytes in the sample, white blood cell population data (e.g., number and cell volume of neutrophils, monocytes, granulocytes, basophils). Besides this, all anemia parameters were analyzed: serum iron level, serum ferritin level, level of vitamin B12, serum folate level, red blood cell folate, serum erythropoietin level, transferrin level, C-reactive protein level, and level of intrinsic factor antibody.

All data were obtained for both groups—anemia patients and patients without anemia. Different groups of patients for analysis as described below.

Groups for descriptive statistics included the following:

(a) Non anemic patients
Hb>12 g/dl, B12<145 pg/ml—Non-anemic, Low B12 (3 patients
Hb>12 g/dl, 145 pg/ml≦B12<180 pg/ml—Non-anemic, Intermediate B12 (2 patients)
Hb>12 g/dl, B12≧180 pg/ml (patients with Low Folate or low RBC folate were excluded from this group and patients with high IFAb (IFAb>1.53 AU/ml) were excluded from this group)—Non-anemic, Normal B12 (48 patients).
Hb>12 g/dl, low Folate (Folate<2.33 ng/ml or Folate RBC<237 ng/ml)—Non-anemic, Low Folate (2 patients)
Hb>12 g/dl, normal Folate (Folate≧2.33 ng/ml and Folate RBC≧237 ng/ml; patients with low or intermediate B12 were excluded from this group and patients with high IFAb (IFAb>1.53 AU/ml) were excluded from this group)—Non-anemic, Normal Folate (48 patients).

(b) Anemic patients
Hb<12 g/dl, B12<145 pg/ml—Anemic, Low B12 (14 patients).
Hb<12 g/dl, 145 pg/ml≦B12≦180 pg/ml—Anemic, Intermediate B12 (4 patients).
Hb<12 g/dl, B12≧180 pg/ml—Anemic, Normal B12 (62 patients). From this group were excluded:
   i. Patients with Low Folate (Folate<2.33 ng/ml) or low RBC Folate (Folate RBC<237 ng/ml).
   ii. Patients with very high B12>940 pg/ml—who obviously were on treatment with B12 because of B12 deficiency)
   iii. Patients with high IFAb—IFAb>1.53 AU/ml
Hb<12 g/dl, low Folate (Folate<2.33 ng/ml or Folate RBC<237 ng/ml)—Anemic, Low Folate (17 patients).
Hb<12 g/dl, normal Folate (Folate≧2.33 ng/ml and Folate RBC≧237 ng/ml)—Anemic, Normal Folate (62 patients). From this group were excluded:
   i. Patients with low or intermediate B12 (B12<180 pg/ml)
   ii. Patients with very high B12>940 pg/ml—who obviously were on treatment with B12 because of B12 deficiency)
   iii. Patients with high IFAb—IFAb>1.53 AU/ml
Hb<12 g/dl, Low Folate (Folate<2.33 ng/ml or Folate RBC<237 ng/ml) or Low B12 (B12<180 pg/ml)—Anemic, Low Folate or Low B12 (30 patients).
Hb<12 g/dl, other anemias (62 patients)—normal Folate (Folate≧2.33 ng/ml and RBC Folate≧237 ng/ml) and normal B12 (B12≧180 pg/ml). Patients with high IFAb (IFAb>1.53 AU/ml) and patients with B12>940 pg/ml (on treatment with B12) were excluded from this group.
   Normals—Hb≧12 g/dl, B12≧180 pg/ml, Folate≧2.33 ng/ml, Folate RBC≧237 ng/ml, CRP<0.5 mg/dL, IFAb<1.53 AU/ml, Ferritin>22 ng/ml (29 patients).

Additional groups for descriptive statistics included:
Normals—Hb≧12 g/dl, B12≧180 pg/ml, Folate≧2.33 ng/ml, Folate RBC≧237 ng/ml, CRP<0.5, IFAb<1.53 AU/ml, Ferritin>22 ng/ml (29 patients)
B12 Deficiency & Interm—B12<180 pg/ml, no selection on Hb, both anemia and non-anemic were included in this group (23 patients)
B12 Deficiency—B12<145 pg/ml, no selection on Hb, both anemia and non-anemic were included in this group (17 patients)
Folate Deficiency—Folate<2.33 ng/ml or Folate RBC<237 ng/ml, no selection on Hb, both anemia and non-anemic were included in this group (19 patients)
B12 or Folate Deficiency—Folate<2.33 ng/ml or Folate RBC<237 ng/ml, or B12<180 pg/ml, no selection on Hb, both anemia and non-anemic were included in this group (36 patients)
B12 Low, Anemia—Hb<12 g/dl, B12<145 pg/ml (14 patients)
B12 def (Low and Interm), Anemia—Hb<12 g/dl, B12<180 pg/ml (18 patients)
Folate def, Anemia—Hb<12 g/dl, Folate<2.33 ng/ml or Folate RBC<237 ng/ml (17 patients)
B12 (Low and Intern) or Folate def—Hb<12 g/dl, B12<180 pg/ml or Folate<2.33 ng/ml or Folate RBC<237 ng/ml (30 patients).
Other anemias—Hb<12 g/dl, B12≧180 pg/ml, Folate≧2.33 ng/ml and Folate RBC≧237 ng/ml. From this group patients with high IFAb>1.53 AU/ml, and B12>940 pg/ml were excluded (62 patients)

Normal distribution for all parameters in all groups for comparative statistics was analyzed with Kolmogorov-Smirnov test using MedCalc software (MedCalc Software, Mariakerke, Belgium).

Data were analyzed and statistical significance was determined using Mann-Whitney U test and Student's t-test, as appropriate. The Receiver Operating Characteristic (ROC) curves and Box and Whisker Plot graphs were determined using MedCalc software (MedCalc Software, Mariakerke, Belgium), as described in Example 1.

Table 3 provides descriptive statistics for the different groups of patients and normal subjects. The values are the mean of the parameters for the group of n patients.

| | | | | | | |
|---|---|---|---|---|---|---|
| B12/Folate | Category | N | MCV | MNEV | MMOV | B12 or Folate |

Non anemic patients

| | | | | | | |
|---|---|---|---|---|---|---|
| B12 Hb > 12 | Low B12 | 3 | 105.9 | 150.3 | 180 | B12 90 |
| B12 Hb > 12 | Interm B12 | 2 | 86.7 | 141.2 | 168.4 | B12 164 |
| B12 Hb > 12 | Normal B12 | 48 | 91.4 | 138.4 | 163.3 | B12 372.6 |
| Folate Hb > 12 | Low Folate | 2 | 105.5 | 147.9 | 170.2 | Folate < 2.33 |
| Folate Hb > 12 | Normal Folate | 48 | 91.4 | 138.4 | 163.3 | Folate 5.8 |

Anemic patients

| | | | | | | |
|---|---|---|---|---|---|---|
| B12 Hb < 12 | Low B12 | 14 | 94.9 | 152.9 | 178.8 | B12 95.9 |
| B12 Hb < 12 | Interm B12 | 4 | 87.1 | 144.1 | 177.8 | B12 164.8 |
| B12 Hb < 12 | Normal B12 | 62 | 84.6 | 142.5 | 169 | B12 355.1 |
| Folate Hb < 12 | Low Folate or | 17 | 93.5 | 151.7 | 178.1 | Folate 1.9 |
| | RBC Folate | | | | | |
| Folate Hb < 12 | Normal Folate | 62 | 84.6 | 142.5 | 169 | Folate 355.1 |
| B12 and | Low Folate or | 30 | 91.6 | 151 | 176.9 | |
| Folate Hb < 12 | B12 | | | | | |
| B12 and | Other anemias | 62 | 84.6 | 142.5 | 169 | |
| Folate Hb < 12 | | | | | | |

Normals

| | | | | |
|---|---|---|---|---|
| Normals | 29 | 92.1 | 138.5 | 162.1 |

Table 4 below illustrates comparative statistics between the different groups of normals and patients with or without Folate or B12 deficiencies. As shown above, the cut-off values obtained from the ROC analysis are used as the criterion for indication of a vitamin B12 and/or folate deficiency.

Comparative Statistics

| N | Parameter | T-Test | ROC AUC | Sens. | Specif. | Cut Off | Significance ROC |
|---|---|---|---|---|---|---|---|
| 23 vs 29 | B12 Def & Interm vs Normals | | | | | | |
| | MCV | p = 0.477 | 0.475 | 39.10% | 100% | >99.5 | p = 0.8154 |
| | NE Mean Volume | p < 0.0001 | 0.826 | 82.60% | 75.90% | >140.318 | p = 0.0001 |
| | Mo Mean Volume | p < 0.0001 | 0.895 | 87.00% | 86.20% | >167.11 | p = 0.0001 |
| 17 vs 29 | B12 Def vs Normals | | | | | | |
| | MCV | p = 0.1906 | 0.615 | 52.90% | 100% | >99.5 | p = 0.3223 |
| | NE Mean Volume | p < 0.0001 | 0.844 | 70.59% | 93.10% | >145.4 | p = 0.0001 |
| | Mo Mean Volume | p < 0.0001 | 0.905 | 88.24% | 86.21% | >167.11 | p = 0.0001 |
| 19 vs 29 | Folate Def (Folate Low or RBC Folate Low) vs Normals | | | | | | |
| | MCV | p = 0.3322 | 0.591 | 52.60% | 86.20% | <89.5 | p = 0.3761 |
| | NE Mean Volume | p < 0.0001 | 0.844 | 89.50% | 75.90% | >140.318 | p = 0.0001 |
| | Mo Mean Volume | p < 0.0001 | 0.878 | 73.70% | 93.10% | >169.5 | p = 0.0001 |
| 36 vs 29 | B12 or Folate deficiency (Folate Low or RBC Folate Low) vs Normals | | | | | | |
| | MCV | p = 0.8622 | 0.602 | 55.6 | 86.2 | <89.5 | p = 0.1803 |
| | NE Mean Volume | p < 0.0001 | 0.822 | 83.3 | 75.9 | >140.318 | p = 0.0001 |
| | Mo Mean Volume | p < 0.0001 | 0.875 | 80.6 | 86.2 | >167.11 | p = 0.0001 |
| 14 vs 62 | B12 Low, anemia vs Other Anemias | | | | | | |
| | MCV | p = 0.0067 | 0.657 | 50.00% | 98.40% | >99.7 | p = 0.1396 |
| | NE Mean Volume | p = 0.0001 | 0.735 | 85.70% | 54.10% | >143.1 | p = 0.0025 |
| | Mo Mean Volume | p = 0.0012 | 0.689 | 64.30% | 66% | >172.35 | p = 0.0204 |
| 18 vs 62 | B12 Def (Low + Intermediate), Anemia vs Other Anemias | | | | | | |
| | MCV | p = 0.0108 | 0.634 | 38.90% | 98.40% | >99.7 | p = 0.1242 |
| | NE Mean Volume | p = 0.0005 | 0.699 | 66.70% | 67.20% | >146.053 | p = 0.0040 |
| | Mo Mean Volume | p = 0.0004 | 0.723 | 172.20% | 66% | >172.35 | p = 0.0011 |
| 17 vs 62 | Folate Def (Folate or rFolate Low), Anemia vs Other Anemias | | | | | | |
| | MCV | p = 0.0046 | 0.662 | 82% | 46.80% | >85 | p = 0.0251 |
| | NE Mean Volume | p = 0.0002 | 0.711 | 88.20% | 49.20% | >141.3 | p = 0.0020 |
| | Mo Mean Volume | p = 0.0009 | 0.691 | 76.50% | 54.10% | >169.7 | 0.0099 |
| 30 vs 62 | B12 (Low&Interm) or Folate deficiency, Anemia vs Other Anemias | | | | | | |
| | MCV | p = 0.0102 | 0.611 | 30 | 96.8 | >96.9 | p = 0.0894 |
| | NE Mean Volume | p = 0.0018 | 0.702 | 86.7 | 49.2 | >141.34 | p = 0.0004 |
| | | (Mann Whitney) | | | | | |
| | Mo Mean Volume | p = 0.0005 | 0.681 | 83.3 | 42.6 | >166.91 | p = 0.0023 |

Results indicated that 5.2% of the “normal” patients with a normal hemoglobin level had a vitamin B12 deficiency (vitamin B12<145 pg/mL); 3.5% of the “normal” patients with a normal hemoglobin level had intermediate values of vitamin B12 (vitamin B12>145<180 pg/mL); and 3.5% of the “normal” patients with a normal hemoglobin level had low serum folate.

From the group of anemic patients, results indicated that the prevalence of vitamin B12 deficiency was 14/109 (12.8%), the prevalence of intermediate vitamin B12 levels was 4/109 (3.7%), the presence of serum folate deficiency was 13/109 (11.9%), the prevalence of vitamin B12 and serum folate deficiency was 1/109 (0.9%), and the prevalence intermediate vitamin B12 levels and serum folate deficiency was 1/109 (0.9%).

Results indicated that after comparing the hematological tests of control subjects with the tests of patients with a vitamin B12 deficiency, MMOV was more accurate in detecting samples with the B12 deficiency followed by the MNEV. When comparing patients with vitamin. B12 or folate deficiencies with patients with anemia (without such deficiencies), the most accurate test was the MNEV, followed by the MMOV. MMOV was determined to be the best parameter for detecting latent vitamin B12 deficiency in a sample.

The data presented in Table 5 indicate that the MNEV and the MMOV can detect B12 or Folate deficiencies in patients with or without anemia with normal MCV. In all cases presented in Table 5 with normal MCV we can detect B12 and/or Folate deficiency with MNEV and/or MMOV.

Table 5 below shows examples of data for patients with B12 and/or Folate deficiencies with and without anemia. (Each different patient is in different columns; grey color shows when B12 or Folate deficiencies are detectable with MMOV or MNEV). All patients have normal or even low MCV and they cannot be detected as B12/Folate deficient with “classical” parameter. In all cases B12 and/or folate deficiency can be detected with MNEV and/or MMOV.

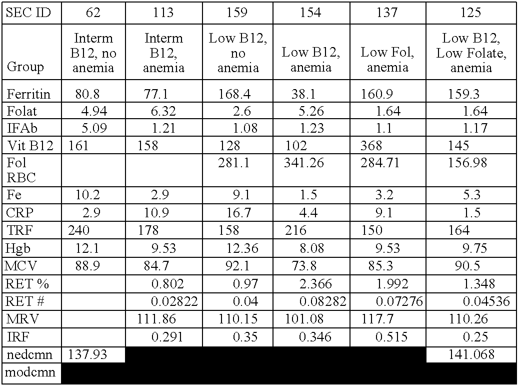

The data presented herein indicates that the MNEV and MMOV have significantly higher sensitivity for the detection of vitamin B12 and/or folate deficiencies in a sample compared to MCV. It should be noted that MNEV and MMOV are not affected by iron deficiency or other causes of anemia, unlike MCV.

Unless defined otherwise in this specification, technical and scientific terms used herein have the same meaning as commonly understood by one of ordinary skill in the art to which this invention belongs and by reference to published texts. All documents listed in this specification and the disclosure of U.S. provisional application No. 61/112,499, are incorporated herein by reference. While the invention has been described with reference to specific embodiments, it is appreciated that modifications can be made without departing from the spirit of the invention. Such modifications are intended to fall within the scope of the appended claims.

What is claimed is:

1. A method of detecting a vitamin B12 or folate deficiency, or monitoring the treatment of the deficiency, in a blood sample from a mammalian subject comprising
comparing a cell volume parameter of a test blood sample from a mammalian subject to a predetermined criterion, the cell volume parameter being obtained from a cell volume distribution of a white blood cell population in the test blood sample,
wherein a vitamin B12 or folate deficiency thereof in the individual is detected or indicated by a difference between the cell volume parameter and the predetermined criterion.

2. The method of claim 1, wherein the predetermined criterion is a cell volume parameter obtained from a white blood cell population of a plurality of control blood samples from subjects selected from the group consisting of
(a) subjects known to be deficient in vitamin B12 and/or folate;
(b) healthy subjects;
(c) subjects having a disease or disorder related to deficiencies in vitamin B12 and/or folate; and
(d) a temporally-earlier test blood sample of the same subject.

3. The method of claim 2, comprising detecting the deficiency when the cell volume parameter is statistically similar to the predetermined criterion of (a) or (c).

4. The method of claim 2, comprising detecting the deficiency when the cell volume parameter is statistically different from the predetermined criterion of (b).

5. The method of claim 2, comprising detecting a latent deficiency when the cell volume parameter is statistically different from the predetermined criterion of (b) and approaching the predetermined criterion of (a) or (c).

6. The method of claim 2, further comprising detecting an improvement in said deficiency in the course of treatment therefor when the difference in the cell volume parameter and (b) is statistically closer than the difference in (d) and (b).

7. The method of claim 1, wherein the white blood cell population comprises leukocytes.

8. The method of claim 1, wherein the white blood cell population comprises a member selected from the group consisting of neutrophils, monocytes or both neutrophils and monocytes.

9. The method of claim 1, further comprising measuring the cell volume parameter obtained from a cell volume distribution of a white blood cell population in the test blood sample prior to the comparison.

10. The method of claim 1, wherein the cell volume parameter is selected from the group consisting of a mean of the cell volume distribution, a standard deviation of the cell volume distribution, an impedance measurement from the sample, a low angle light scatter measurement from the sample and an axial light loss measurement from the sample.

11. The method of claim 10, wherein the cell volume parameter is mean neutrophil volume, mean monocyte volume, or both parameters.

12. The method of claim 1, wherein the test blood sample is a whole blood sample.

13. The method of claim 1, wherein the method further comprises mixing the test blood sample with a lytic reagent system.

14. The method of claim 1, which is performed by a computer processor or computer-programmed instrument that generates numerical or graphical data useful in detecting the deficiency.

15. The method of claim 1, wherein the comparing step comprises at least one of:
comparing a cell volume parameter obtained from a cell volume distribution of a first white blood cell subpopulation of neutrophils in the test blood sample to a first predetermined criterion obtained from a white blood cell subpopulation of neutrophils from a plurality of control blood samples; and
comparing a cell volume parameter obtained from a cell volume distribution of a second white blood cell subpopulation of monocytes in the test blood sample to a second predetermined criterion obtained from a white blood cell subpopulation of monocytes from a plurality of control blood samples.

16. The method of claim 1, further comprising comparing a cell volume parameter obtained from a cell volume distribution of a red blood cell population in the test blood sample to a predetermined criterion obtained from a red blood cell population from a plurality of control blood samples.

17. The method of claim 16, wherein the cell volume parameter of the red blood cell population is selected from the group consisting of a mean of the red blood cell volume distribution, a standard deviation of the red blood cell volume distribution, an impedance measurement, a low angle light scatter measurement and an axial light loss measurement, and a mean of the cell volume distribution.

18. The method of claim 1, further comprising coupling the comparative difference in the cell volume parameter of the test blood sample and the predetermined criterion with the presentation of clinical symptoms of the B12 or folate deficiency in the subject.

19. The method of claim 1, which provides a quantitative assessment of the likelihood or risk of B12 deficiency or folate deficiency occurrence in a subject that has not yet developed clinical symptoms of the deficiency.

20. A method of detecting a vitamin B12 or folate deficiency, or monitoring the treatment of the deficiency, in a blood sample from a mammalian subject comprising comparing a parameter selected from the group consisting of mean neutrophil volume and mean monocyte volume of a test blood sample from a mammalian subject to a predetermined criterion,
wherein a vitamin B12 or folate deficiency thereof in the individual is detected or indicated by a difference between the parameter and the predetermined criterion.
